(19) United States
(12) Patent Application Publication
(10) Pub. No.: US 2015/0099936 A1
(43) Pub. Date: Apr. 9, 2015

(54) SHEATH SUPPORT DEVICES, SYSTEMS AND METHODS

(71) Applicant: SPINAL MODULATION, INC., Menlo Park, CA (US)

(72) Inventors: Albert G. BURDULIS, San Francisco, CA (US); Evan S. VANDENBRINK, San Francisco, CA (US); Fred I. LINKER, Los Altos, CA (US)

(21) Appl. No.: 14/510,338

(22) Filed: Oct. 9, 2014

Related U.S. Application Data
(60) Provisional application No. 61/888,900, filed on Oct. 9, 2013.

(51) Int. Cl. A61M 25/01; A61B 1/32; A61M 5/14; A61B 1/005; A61M 25/06; A61B 5/00; A61N 1/36; A61B 18/00; A61B 1/313; A61M 25/09; A61N 1/05

(52) U.S. Cl. CPC A61M 25/0194; A61M 25/09; A61B 1/32; A61M 5/14; A61B 1/005; A61N 1/0551; A61B 5/6852; A61N 1/36071; A61B 18/00; A61B 1/3132; A61M 25/065; A61B 2018/00577
USPC 600/204; 604/264; 604/523; 604/506; 606/129

(57) ABSTRACT

Devices, systems and methods are provided for accessing a target location in the body of a patient, particularly within the epidural space. A system includes a sheath and a sheath support supporting the sheath to reduce or avoid kinking. The sheath support closely fits within the sheath while maintaining free sliding therein. The sheath support has a non-compliant outer diameter maintaining the inner diameter of the sheath and preventing the sheath walls from collapsing into a kink, particularly during low radius bends that may occur during delivery. The sheath support may include a distal tip configured to resist retraction into the sheath until a threshold force is reached which causes the distal tip to at least partially retract into the lumen of the sheath. Likewise, the distal tip may be fully retractable through the sheath so that the sheath support is removable from the proximal end of the sheath.

### CROSS-REFERENCES TO RELATED APPLICATIONS

This application claims the benefit of U.S. Provisional Application No. 61/888,900, filed Oct. 9, 2013, which application is incorporated herein by reference.

### BACKGROUND OF THE INVENTION

Chronic pain is a condition that has proven to be challenging to the physician, patient, and society. Successful and long-lasting treatment can be difficult to attain. The algorithmic treatment of suffering often starts with simple interventions such as physical medicine and non-steroidal medications and then progresses to complex interventions. Near the end of the algorithm, the use of spinal cord stimulation (SCS) has been employed to treat those with complex pain. Conventional SCS is often used to treat chronic, intractable pain when other therapies have failed. SCS has been shown to be effective in some patients having a variety of neuropathic pain conditions. However, despite its clinical utility for some patients, SCS therapy carries limitations. Twenty percent of subjects trialing an SCS system do not proceed beyond the trial stimulation. Overall, the treatment has been found to be a successful long-term solution in approximately 50% of patients that have a successful temporary trial stimulation. Failures may be due to difficulty in programming the device to align the stimulation-associated paresthesias with the painful areas of the body, inability to derive the correct combination of pulse width, frequency, and amplitude of the electrical waveform needed to address the individual's pain, or due to device issues such as lead migration. Conventional SCS can also be vulnerable to positional or postural effects in which the intensity or location of paresthesias may change when the subject changes his/her body position such as moving from lying to sitting. This change is due to shifts in the relative distance between the stimulating electrodes and the dorsal columns through the effects of gravity or physical forces due to epidural lead placement in the highly mobile spine. Additionally, some patients may not tolerate the pins-and-needles sensation of the paresthesias associated with SCS, particularly if these are extraneous and located in nonpainful areas of the body.

Consequently, other forms of treatment have been investigated. For example, neuromodulation of the dorsal root ganglion is a newly developed treatment for chronic pain and other conditions. Recent advances in the understanding of the role that the dorsal root ganglion plays in both the development and maintenance of chronic pain and in other conditions have significantly advanced over the past several years. The DRG contains the cell bodies (ie, somata) for most of the primary sensory neurons (PSNs) and is therefore a critical structure in the transmission and transduction of pain. Depending on its level in the spinal column, a DRG may contain up to 15,000 PSNs. Primary sensory neurons are bipolar, also referred to as pseudounipolar, meaning that each cell has a single stem axon, which extends a short distance from its soma via its axon hillock and then splits into 2 long branches. This bifurcation at the “T-junction” gives rise to the peripheral branch, which conveys sensory input from the periphery to the DRG cell body, and the central branch, which carries information from the DRG cell body to the spinal cord via the dorsal roots. The primary sensory neurons transduce information from a variety of receptors, including nociceptors, thermoreceptors, chemoreceptors, and proprioceptors, via varied nerve sizes: C-fibers, A-delta, and A-beta types, divided into the dorsal column-medial lemniscus system (touch/proprioception/vibration), the anterolateral system (somatic pain/temperature), and the postsynaptic dorsal column system (visceral pain). Thus, the DRG has been described as the “gatekeeper” for the primary afferent nerves, and accordingly, the dorsal root ganglion has become a neuromodulation point for therapy.

The dorsal root ganglion is located along the dorsal root which is one of the spinal nerves extending from the spinal cord. The spinal nerves include both dorsal and ventral roots which fuse in or near the intervertebral foramen to create a mixed nerve which is part of the peripheral nervous system. At least one dorsal root ganglion (DRG) is disposed along each dorsal root prior to the point of mixing. There are 7 paired cervical DRGs, 12 paired thoracic DRGs, 5 paired lumbar DRGs and 4 paired sacral DRGs in humans. The DRG is located near the distal end of the dorsal root in the lateral epidural space and is typically within the intervertebral foramen. Thus, the DRGs reside behind the vertebral artery and are protected from external physical stimuli by the vertebrae.

Systems and devices have been developed to stimulate one or more dorsal root ganglia. Accessing these areas is challenging, particularly from an antegrade epidural approach. FIG. 1 schematically illustrates portions of the anatomy in such areas. As shown, each DRG is disposed along a dorsal root DR and typically resides at least partially between the pedicles PD or within a foramen. Each dorsal root DR exits the spinal cord S at an angle θ. This angle θ is considered the nerve root sleeve angulation and varies slightly by patient and by location along the spinal column. The average nerve root angulation in the lumbar spine is typically less than 90 degrees and more typically less than 45 degrees. Therefore, accessing this anatomy from an antegrade approach involves making a sharp turn through, along or near the nerve root sleeve angulation.

FIG. 1 illustrates a lead 10 inserted epidurally and advanced in an antegrade direction along the spinal cord S. The lead 10 having electrodes 12 thereon is advanced through the patient anatomy so that the electrodes 12 are positioned on the target DRG. Such advancement of the lead 10 toward the target DRG in this manner involves making a sharp turn along the angle θ. In some instances, such a turn can be achieved with the use of leads and delivery tools described in U.S. patent application Ser. No 12/687,737, entitled “Stimulation Leads, Delivery Systems and Methods of Use”, assigned to the present applicant and incorporated herein by reference.

Despite the current success of DRG neuromodulation, improved devices, systems and methods are desired. The economic burden on society from the detrimental effects of chronic pain and other chronic and debilitating conditions is ever increasing, and recent numbers bear the financial impact. In light of a need for increasing therapeutic options, advances in neuromodulation of the DRG and other anatomies are continuously desired. At least some of these needs are met by the present invention.

### BRIEF SUMMARY OF THE INVENTION

The present invention relates to medical devices, systems, and methods. In particular, the present invention relates to devices, systems, and methods for delivering or implanting one or more neuromodulation devices in the body of a patient. The devices, systems, and methods disclosed herein may find particular use for neuromodulation of the central nervous system, including the spinal cord and spinal nerves. In most embodiments, neuromodulation comprises stimulation, however it may be appreciated that neuromodulation may include a variety of forms of altering or modulating nerve activity, such as by delivering electrical and/or pharmaceutical agents.

A variety of improvements in the field of neuromodulation are provided, particularly in the delivery of devices to target anatomies within the spinal anatomy, more particularly in the delivery of devices to one or more dorsal root ganglions. Although sheaths have been used to deliver leads within the epidural space, kinking of such sheaths during delivery is an issue in some circumstances. In some instances, advancement of a sheath alone or with a compliant device, such as a floppy lead, therein can allow kinks to form in the sheath. This can occur during introduction through the needle entering the epidural space (due to mishandling of the distal tip of the sheath) or during deployment of the sheath into the epidural space (due to the distal end of the sheath encountering bone or other tissue that causes the sheath to undergo significant bending). Once the sheath is kinked, deployment of the device therein is hindered. In some instances, such hindering completely obstructs deployment. This is because the sheath kink reduces the inner diameter of the sheath, pinching the device in place. Likewise, kinking can make removal of the sheath through the needle difficult. In some instances, the sheath kink has increased the outer diameter of the sheath, binding the sheath in the needle.

The present invention provides, among others, a sheath support which supports the sheath to reduce or avoid kinking. The sheath support closely fits within the sheath while maintaining free sliding therein. Thus, the sheath support has a non-compliant outer diameter that maintains the inner diameter of the sheath and prevents the sheath walls from collapsing into a kink, particularly during low radius bends that may occur during delivery within the body. Such a design is particularly useful during insertion through a needle and advancement within the epidural space, particularly when approaching a target dorsal root ganglion (DRG).

The sheath support of the present invention allows the sheath to have a lower stiffness and/or wall thickness than would be otherwise possible while avoiding kinking. Thus, the sheath support avoids the need for sheath wall reinforcement, such as braiding, which may increase stiffness and/or wall thickness. The sheath support also resists kinking in a manner that is typically superior to sheath wall reinforcement. Thus, use of the sheath support allows the sheath to be softer, more flexible, and/or include other variations of construction, geometry and materials.

In addition to reduced kinking, the sheath support of the present invention provides a variety of other benefits. For example, different types of sheath supports may be used to deliver the same sheath depending upon the situation, such as the anatomy or procedure. Sheath support variations include straight, pre-curved, various pre-curved tips (e.g. large, small, short, long etc.), high flexibility, low flexibility, to name a few. Likewise, the sheath support may be removed and replaced at any point during the procedure, leaving the sheath in place during the transaction.

The sheath support may also have a variety of distal tips. In some embodiments, the sheath support has a special-use tip. For example, the sheath support may have a cutting tip to assist in advancing the sheath and sheath support through scar tissue or other obstacles within the epidural space. Other types of special-use tips include drug or agent delivery tips, vision tips, electrical energy delivery tips, stimulation tips, etc. In other embodiments, the sheath support has an atraumatic distal tip, such as a rounded, ball-shaped tip. Such a tip covers and protects the distal end of the sheath while simultaneously allowing the sheath and sheath support to advance through tissue in an atraumatic manner. It may be appreciated that the sheath support may be removed to exchange the sheath support, or in some embodiments the tip itself, at any point during the procedure, leaving the sheath in place during the transaction.

Use of the sheath support also assists in increasing speed and convenience of device delivery to a target location. For instance, positioning a sheath along with the sheath support therein within the body allows for increased steerability and maneuverability due to the support provided by the sheath support. In addition, once the sheath and sheath support are desirably positioned, the sheath may be withdrawn or removed and a new or different sheath may be advanced to the same location over the previously positioned sheath support. Likewise, once the sheath and sheath support are desirably positioned, the sheath support may be withdrawn or removed and a new or different sheath support (such as one with a different tip) may be advanced to the same location within the previously positioned sheath. Similarly, the sheath support may be withdrawn or removed and a lead or tool may be advanced to the same location within the previously positioned sheath. When the sheath support has a distal tip which is larger than the distal tip of the sheath, such as an atraumatic ball-shaped distal tip, the distal tip of the sheath support may resist retraction into the sheath until a threshold force is reached which causes the distal tip to at least partially retract into the lumen of the sheath. In some instances, the distal tip at least partially collapses to be at least partially retracted within the sheath. In some embodiments, the distal tip may be completely retracted into the sheath, such as for removal out of the proximal end of the sheath. Such removal allows the sheath to remain in its desired position for delivery therethrough of a lead, catheter, stylet, guidewire or other tool. Otherwise, the sheath and sheath support would need to be removed to allow the sheath support to be removed distally which would require repositioning of the sheath within the body. Thus, procedure time is reduced and ease of use is increased.

Once the sheath is desirably positioned, a variety of tools, devices or leads may be advanced therethrough to the target location. Thus, the sheath forms a conduit to the target location and allows exchange of tools, devices or leads via the proximal end of the sheath. Typically, a lead is advanced through the sheath to the target location, such as a dorsal root ganglion. Since the lead is guided through the sheath, the lead may be very flexible and floppy. Such leads are desirable for positioning within the epidural space since they allow greater movement and flexibility when the patient moves and goes about daily life. Highly flexible leads are also more easily placed in restricted and/or high movement areas of the body. Further, highly flexible leads can be anchored by creating a slack anchor, such as described in U.S. patent application Ser. No. 13/104,787, entitled “Methods, Systems and Devices for Anchoring in the Epidural Space”, incorporated herein by reference. By separating the features needed for efficient and desirable delivery of the sheath from the features needed for desirable functionality of the lead, each device may be optimized for performance regardless of conflicting needs.

In a first aspect of the present invention, a system is provided for accessing a target location in the epidural space of a patient. The system comprises a sheath having a proximal end, a pre-curved distal end and a lumen having an inner diameter, and a sheath support having a shaft configured to be disposed within the lumen of the sheath and a distal tip. The sheath support is sufficiently flexible to bend according to the pre-curved distal end of the sheath and the sheath support has a non-compliant outer diameter that maintains the inner diameter of the lumen of the sheath so as to resist kinking of the sheath.

In some embodiments, the distal tip of the sheath support is retractable through the lumen of the sheath and removable from the proximal end of the sheath. Typically, together the sheath and sheath support disposed therein is flexible. As such, the sheath and sheath support disposed therein is often advanceable through an epidural introducing needle.

In some embodiments, the sheath support shaft has an outer diameter that sufficiently matches the inner diameter of the sheath while allowing movement of the sheath support relative to the sheath.

In some embodiments, the sheath support shaft is comprised of a coil. In some embodiments, a distal portion of the coil has a larger pitch than a proximal portion. In other embodiments, the distal tip comprises a distal end cap molded to the coil. In some instances, the distal end cap comprises an inner tubular shaft, outer tubular shaft, and a tip piece, wherein the inner and outer tubular shafts are concentrically positioned and joined at one end by the tip piece. Typically, the distal end cap is positioned over a distal end of the coil so that the coil is disposed between the inner tubular shaft and the outer tubular shaft. The tip piece may then cover the distal end of the coil.

In some embodiments, the system further comprises an elongate device adapted to be advanced through the sheath such that the curved distal portion of the sheath bends and guides the elongate device toward the target location as the elongate device is advanced therethrough. Example elongate devices include a lead, catheter, stylet, guidewire, or tool, to name a few.

In some embodiments, the target location comprises a spinal nerve. Optionally, the target location may comprise a dorsal root ganglion.

In some embodiments, the system further comprises a retraction shield having a lumen, wherein the retraction shield is configured to be disposed within the lumen of the sheath while the sheath support shaft is disposed within the lumen of the retraction shield. In some embodiments, the distal tip is at least partially retractable into the lumen of the retraction shield and together the sheath support and retraction shield are removable from the proximal end of the sheath.

In some embodiments, the distal tip is configured to resist retraction into the lumen of the sheath until a threshold force is reached which causes the atraumatic distal tip to at least partially retract into the lumen of the sheath. In some instances, the distal tip at least partially collapses while it at least partially retracts into the lumen of the sheath. In these and/or other instances, a portion of the sheath support shaft is configured to at least partially collapse while the distal tip at least partially retracts into the lumen of the sheath. In some embodiments, the distal tip is comprised of a flexible polymer which changes shape while at least partially retracting into the lumen of the sheath. The distal tip may have a ball shape. Or the distal tip may simply have an atraumatic shape. It may be appreciated that in some embodiments, the distal tip has a special-use tip, such as a cutting tip, agent delivery tip, a vision tips, an electrical energy delivery tip, and/or a stimulation tip.

In another aspect of the present invention, a method is provided for accessing a target location in the epidural space of a patient, the method comprising advancing an introducer needle into the epidural space, advancing a sheath and a sheath support disposed therein through the introducer needle and within the epidural space toward the target location, wherein the sheath support is sufficiently flexible to bend according to a pre-curved distal end of the sheath and wherein the sheath support has a non-compliant outer diameter that maintains the inner diameter of the lumen of the sheath so as to resist kinking of the sheath, positioning the distal end of the sheath and sheath support disposed therein near the target location, and retracting the sheath support into the sheath and removing the sheath support from a proximal end of the sheath leaving the distal end of the sheath near the target location.

Typically, the target location comprises a dorsal root ganglion. In some embodiments, the positioning step comprises positioning the distal end of the sheath and the sheath support disposed therein along a nerve root associated with the dorsal root ganglion. In some embodiments, the positioning step comprises positioning the distal end of the sheath and the sheath support disposed therein within a foramen associated with the dorsal root ganglion.

In some embodiments, the method further comprises inserting an elongate device through the sheath such that at least a portion of the device extends out of the distal end of the sheath toward the target location. The elongate device may comprise a lead, guidewire, stylet, or tool, to name a few. In some instances, the elongate device comprises a lead having at least one electrode and the method further comprises delivering stimulation energy from at least one of the at least one electrode toward the target location. Typically, the target location comprises a dorsal root ganglion.

In other embodiments, the elongate device comprises an agent delivery device and the method further comprises delivering an agent to the target location. Again, typically the target location comprises a dorsal root ganglion.

In some embodiments, the method further comprises advancing the sheath support beyond the distal end of the sheath so that the distal tip atraumatically tunnels through a resistant area of the epidural space. For example, the resistant area may comprise a foramen and the tunneling creates additional space within the foramen. Optionally, the method may include advancing and retracting the sheath support to create additional tunneling force or friction along the resistant area.

In some embodiments, the sheath support has an atraumatic distal tip configured to resist retraction into the sheath while covering a distal end of the sheath, wherein the retracting step further comprises applying a threshold force during retracting which overcomes the resistance allowing the atraumatic distal tip to at least partially retract into the lumen of the sheath. In some instances, the method further comprises advancing an elongate device through the sheath to perform a function at the target location, wherein the function includes neuromodulating, electrically stimulating, sensing, cutting, piercing, ablating, visualizing and/or delivering an agent. It may be appreciated that in some instances the elongate device comprises a lead having at least one electrode and the target location comprises a dorsal root ganglion, the method then further comprises providing stimulation energy to the at least one of the at least one electrodes to selectively stimulate the dorsal root ganglion.

Other objects and advantages of the present invention will become apparent from the detailed description to follow, together with the accompanying drawings.

### BRIEF DESCRIPTION OF THE DRAWINGS

FIG. 1 illustrates a lead inserted epidurally and advanced in an antegrade direction along the spinal cord S.

FIGS. 2A-2B illustrate an embodiment of a sheath having a lumen therethrough and a sheath support which is positionable within the lumen of the sheath.

FIG. 2C illustrates an embodiment of a hub which includes a locking cap which may be used to lock the sheath support in position within the sheath.

FIGS. 3A-3C illustrate an embodiment of a sheath support loaded within a sheath in various positions.

FIGS. 4A-4C illustrate an embodiment of a sheath support of the present invention.

FIGS. 5A-5C illustrate the embodiment of the sheath support of FIGS. 4A-4C being retracted into the sheath.

FIGS. 6A-6C illustrates an embodiment of the sheath support having a distal tip which is comprised of a compliant solid material.

FIG. 7A-7D illustrate another embodiment of a distal tip of a sheath support.

FIGS. 8A-8B illustrate an embodiment of a system including a sheath, a retraction shield and sheath support.

FIG. 9 illustrates an embodiment of an introducing needle accessing the epidural space.

FIG. 10 illustrates attachment of an embodiment of a syringe to the needle of FIG. 9.

FIG. 11 illustrates insertion of a sheath support and sheath inserted through the needle of FIG. 9, into the epidural space.

FIG. 12 illustrates passage of an embodiment of the assembled sheath and sheath support emerging into the epidural space.

FIG. 13 illustrates a cross-sectional view of a vertebrae and spinal column, including the sheath and sheath support of FIG. 12 directed laterally outward, away from the midline of the spinal column, along a dorsal root.

FIG. 14 illustrates an embodiment of a lead advanced through a previously positioned sheath and positioned so that at least one electrode is in proximity to the dorsal root ganglion.

FIG. 15 illustrates an embodiment of a sheath support advanced distally from the distal tip of the sheath.

FIGS. 16A-16B illustrate an embodiment of a lead.

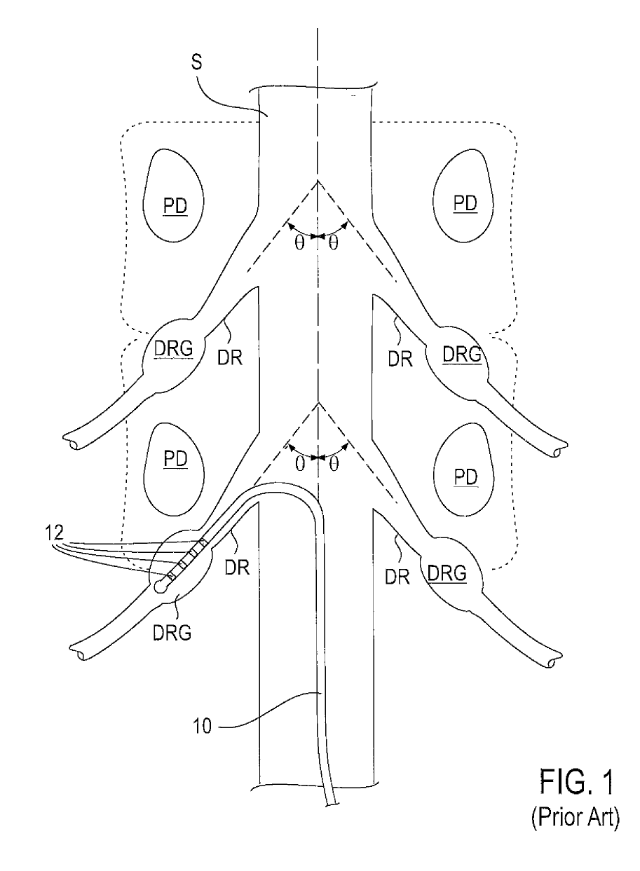

### DETAILED DESCRIPTION OF THE INVENTION

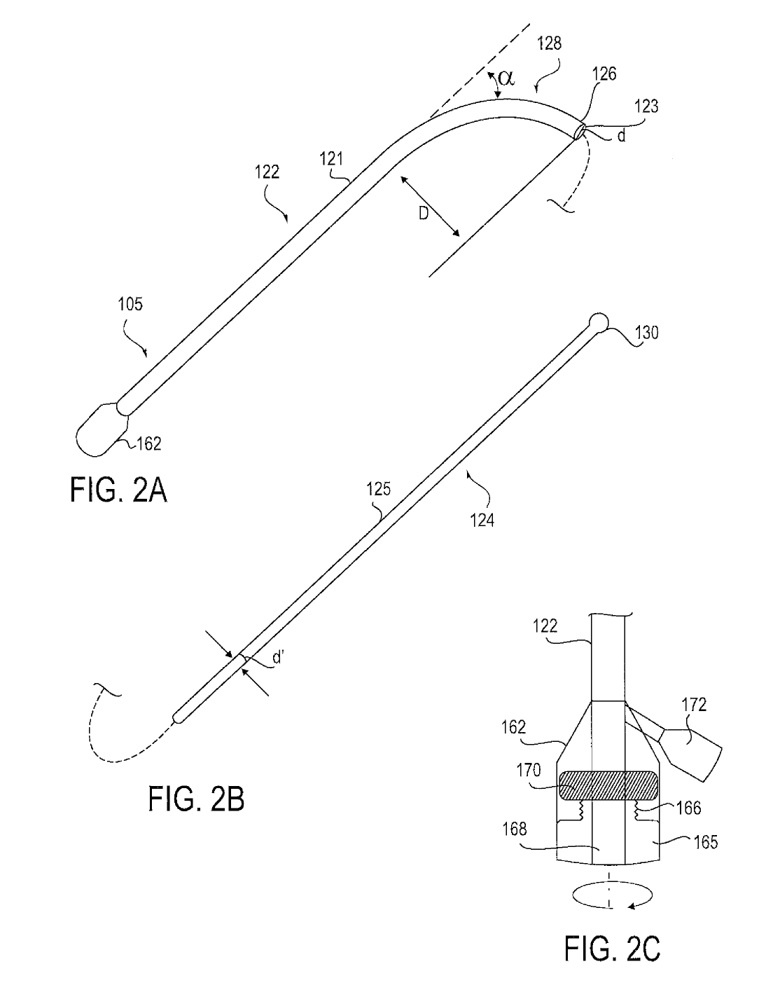

FIGS. 2A-2B illustrate an embodiment of a system for accessing a target location in a body of a patient, particularly a target location in an epidural space of a body of a patient. In particular, FIG. 2A illustrates an embodiment of a sheath 122 having a proximal end 105, a distal end 128, and a lumen 123 therethrough. The lumen 123 has an inner diameter d. FIG. 2B illustrates a sheath support 124 which is positionable within the lumen 123 of the sheath 122. The sheath support 124 comprises a shaft 125 having an outer diameter d′. The sheath support 124 also includes a distal tip 130. Together, the sheath 122 and sheath support 124 are advanceable within the body, particularly within the epidural space.

In this embodiment, the sheath 122 comprises an elongate shaft 121 having a distal portion 128 which is pre-curved to have an angle α. In some embodiments, the angle α is in the range of approximately 5-90 degrees, 15-50 degrees or 20-30 degrees. It may be appreciated that the angle α may optionally be greater, such as greater than 90 degrees, forming a U-shaped or tighter bend. The pre-curve or bend can also be characterized by the lateral distance D from the distal tip 126 to the outer surface of the shaft of the sheath 122, as illustrated in FIG. 2A. In some embodiments, the distance D may be approximately 0.030-0.375 inches. Such curvature causes the sheath support 124 positioned therein to bend in accordance with the pre-curvature of the sheath 122. The curvature can assist in steering the sheath 122 and sheath support 124 assembly along the spinal column and toward a target, such as in a lateral direction toward a dorsal root ganglion (DRG). The curvature may also assist in steering the sheath 122 and sheath support 124 assembly along other anatomical spaces and toward various surgical target sites.

In some embodiments, the sheath 122 is sized and shaped for particular types of delivery, such as antegrade, retrograde, and contralateral approaches, to name a few. In some embodiments, an antegrade sheath (configured for antegrade delivery) has a bend with an angle α of approximately 90-110 degrees and a distance D of approximately 0.325-0.375 inches. Bends having an angle α less than or equal to 150 degrees and a distance D of greater than or equal to 0.225 inches typically improve the ease of delivery when using an antegrade approach to the DRG. In some embodiments, an alternate sheath 122 (configured for retrograde or contralateral delivery) may have a distance D of approximately 0.045-0.095 inches. Bends having a distance D of greater than or equal to 0.030 inches typically improve the ease of delivery when using a retrograde or contralateral approach to the DRG. Typically, the sheath 122 is rigid enough to bend the distal end of the sheath support 124, lead or other tools without the sheath 122 significantly deflecting. Alternatively, the sheath 122 may be more flexible to allow increased steering or guiding through the anatomy. In any case, together the sheath and sheath support disposed therein is flexible, such as sufficiently flexible to be advanced through a needle, such as into the epidural space. And, sufficiently flexible to access a dorsal root ganglion from an antegrade approach, e.g. to bend laterally away from the midline of the spinal cord, along a nerve root, toward a dorsal root ganglion, such as through a nerve root sleeve angulation of less than 90 degrees, typically less than or equal to 45 degrees.

In some embodiments, the sheath 122 is comprised of a polymer, such as polyimide, or polyetheretherketone (PEEK). In some embodiments, the sheath 122 is comprised of a plastic material, such as a thermoset and/or thermoplastic material. Polyimide may be used due to the thinness of its walls while retaining high strength, superior shape memory and shape retention. Polyimide can also be straightened for passage through an introducing needle without kinking. In some embodiments, the sheath 122 is comprised of polyimide material having a wall thickness in the range of approximately 0.002-0.006 inches, more particularly approximately 0.003-0.006 inches. It may be appreciated that other materials may be used so that the resulting sheath has an appropriate stiffness to allow advancement along the epidural space, while having a wall-thickness thin enough to allow passage of the sheath 122 through an introducing needle to the epidural space, and while having a sufficiently low coefficient of friction to allow desirable passage of a lead or other tools therethrough. Examples of other materials potentially meeting these criteria include nylon, polycarbonate, acrylonitrile butadiene styrene (ABS), Polyethylene terephthalate (PET) and Pebax, to name a few.

The sheath support 124 comprises a shaft 125 which fills the lumen 123 within the sheath 122 so as to reduce potential kinking of the sheath 122. Typically, the sheath support 124 has an outer diameter d′ that sufficiently matches the inner diameter d of the sheath 122 while allowing movement of the sheath support 124 relative to the sheath 122. Thus, the sheath support 124 can move within the sheath 122 while filling the lumen 123. In addition, the outer diameter d′ of the sheath support 124 is non-compliant so as to further resist kinking of the sheath 122. Such non-compliance prevents any folds or material of the sheath 122 from entering the lumen 123 thereby preventing their kink formation.

The sheath support 124 is also structured so as to provide additional column strength to the sheath 122 which assists in pushability, steerability and force translation. This allows the sheath 122 to be steered and positioned through or within tortuous and/or confining anatomies that may be inaccessible to the sheath 122 delivered without the sheath support 124 therein. In particular, the additional column strength assists in steering the sheath 124 along a path toward the DRG and optionally within the foramen housing the DRG. This path may be filled with fatty tissue, fluid, blood, fascia, connective tissue and scar tissue which can create resistance to advancement of the sheath 122. Likewise, the tight confines of the foramen can also create resistance to advancement of the sheath 122. The additional column strength provided by the sheath support 124 assists advancement of the sheath 124 therethrough.

In some embodiments, the sheath 122 is comprised of a single stiffness or unidurometer material. This may be most suitable when the sheath 122 and sheath support 124 are introduced together to the epidural space, sharing the delivery workload. However, it may be appreciated that sheath 122 may optionally be comprised of a reinforced polymer, such as a braided polymer, or may be comprised of a construct of various materials. For example, the tip 126 of the sheath 122 may be comprised of a differing material or a thinner material to create a less traumatic or an atraumatic tip. Such a tip may be more flexible than the remainder of the sheath 122 which may provide increased torqueability and pushability. Further it may be appreciated that the sheath 122 may optionally be comprised of a flexible metal or metal/polymer construct.

Delivery of sheath 122 and sheath support 124 together also provides a number of other benefits. For example, preloading of the sheath 122 with the sheath support 124 and simultaneous delivery eliminates multiple steps and complications associated with separate introduction of each device. Further, matching the coaxial shapes of the sheath 122 and sheath support 124 can create steerability and control without the need for stiffening construction and without sacrificing flexibility and profile. In addition, preloading of the sheath 122 with a sheath support 124 having a larger diameter (such as a ball shaped) distal tip 130 can allow the sheath 122 to have a comparatively hard or sharp tip because it is shielded by the atraumatic shape and size of the distal tip 130 of the sheath support 124. Thus, the practitioner may be less concerned with traumatizing surrounding tissue during delivery in comparison to advancing a traditional open-tipped sheath. However, it may be appreciated that the distal end of the sheath 122 may optionally be formed from a soft material, such as Pebax, to create a more atraumatic tip for the sheath 122 itself. In such instances, the sheath 122 may optionally be used with a sheath support 124 without an atraumatic distal tip 130.

A larger diameter distal tip 130 of the sheath support 124 can also provide tactile feedback when retracted against the sheath 122. Such feedback allows the practitioner to tactilely determine the relative position of the sheath support 124 to the sheath 122. It may be appreciated that other mechanisms may be used to register the distal tip 130 of the sheath support 124 against the sheath 122, such as slots, pins, and bands, to name a few. Alternatively, such registering may be achieved near the proximal end of the sheath support 124 and the sheath 122.

In most embodiments, the sheath 122 also includes a hub 162, such as illustrated in FIG. 2C, near its proximal end 105 wherein the hub 162 assists in manipulation of the sheath 122. The torsional rigidity of the sheath 122 allows the sheath 122 to be torqued by rotation of the hub 162. Referring to FIG. 2C, in some embodiments the hub 162 includes a locking cap 165 which may be used to lock the sheath support 124 in position within the sheath 122. Such locking may assist in reducing movement of the sheath support 124 during manipulation of the sheath 122. In one embodiment, the locking cap 165 can have a threaded elongated portion 166 which engages with threads within the hub 162. The locking cap 165 can also have an aperture 168 which aligns with a lumen extending through the sheath 122. The sheath support 124 may be advanceable through the aperture 168 and into the lumen of the sheath 122. When the sheath support 124 is desirably positioned, the sheath support 124 may be locked in place by rotating the locking cap 165 which advances the threaded elongated portion 166 into the hub 162 and compresses a gasket 170. The gasket 170 may be comprised of any flexible material, such a silicone. Compression of the gasket 170 can cause the gasket 170 to engage the sheath support 124, thereby locking the sheath support 124 in place by frictional forces. Optionally, the hub 162 may include an injection port 172 which may be used to inject a desired medium, such as contrast, saline, an agent or other fluids.

In some embodiments, the hub 162 also provides indication of the direction of the bend. This can assist in steering with or without the aid of visualization. In instances where visualization is used, such as fluoroscopy, an embodiment of the sheath 122 may be used which has a radiopaque marker near its distal end 128. Alternatively, the sheath 122 may be marked with radiopaque stripes, such as along the distal end 128 or along the length of the sheath 122. Likewise, the sheath 122 may be marked with radiopaque marker bands, such as tungsten or platinum marker bands, since the wall thickness of the sheath 122 is not limited by the epidural space. Alternatively or in addition, the sheath 122 may be loaded with radiopaque material to provide radiopacity along the distal end 128, the distal tip or along its length. In any case, any suitable radiopaque material may be used, such as tungsten or barium sulfate. In some embodiments, the sheath 122 may be less radiopaque than the sheath support 124 or any tools passed therethrough so that the practitioner can maintain visualization of the sheath support 124 and can visualize the interaction of the sheath 122 and sheath support 124 together. Or, in some embodiments, the sheath 122, sheath support 124 or various tools each have radiopaque markers at their respective ends so that the practitioner is aware of their locations, both within the anatomy and in relation to each other. Visualization may be particularly useful for the methods of the present invention which typically involve manipulation of the devices in three dimensions, such as movement in and out of different planes, as opposed to conventional SCS lead placement which occurs in two dimensions.

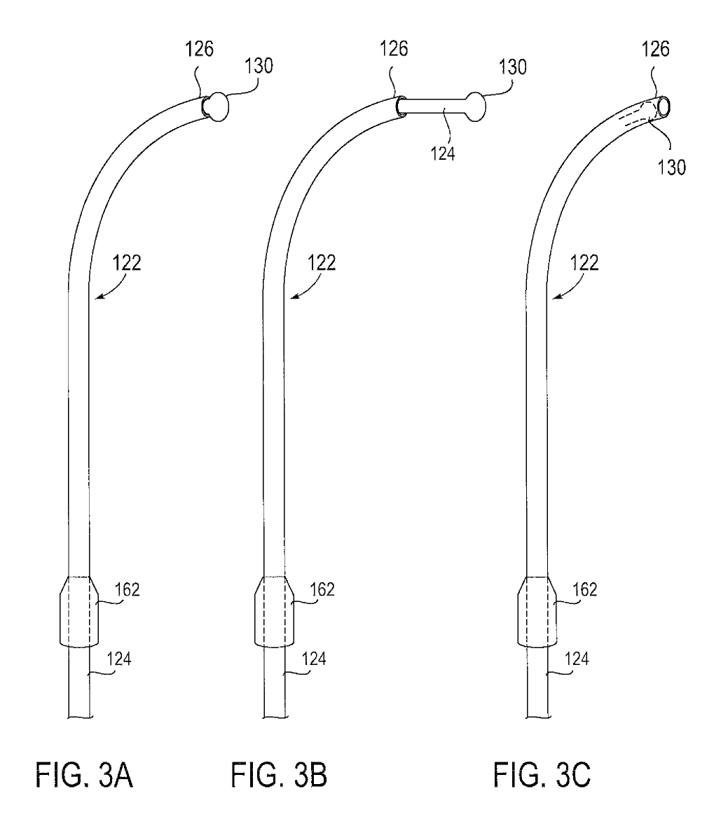

As mentioned previously, in some embodiments, the sheath support 124 has a shaped distal end 130 that is rounded or ball-shaped. Alternatively, the distal tip 130 may instead have a variety of other shapes including a tear drop shape or a cone shape, to name a few. These shapes can provide an atraumatic tip for the sheath support 124 as well as serve other purposes. For example, FIG. 3A illustrates the sheath support 124 loaded within the sheath 122 so that the ball-shaped distal end 130 abuts a distal tip 126 of the sheath 122. In this position, the shaped distal end 130 of the sheath support 124 forms an atraumatic cover for the distal tip 126 of the sheath 122. The sheath support 124 may be advanced, as illustrated in FIG. 3B, so that the distal end of the sheath support 124 extends beyond the distal tip 126 of the sheath 122. Such advancement may be useful in steering the sheath 122 and/or tunneling through tissues ahead of the sheath 122. In some instances, the sheath support 124 may be quickly advanced and retracted to create additional tunneling force or friction. Such tunneling may be useful for further advancement of the sheath 122 and/or later advancement of a lead or other tools through the sheath 122 and into the tunnel.

Referring to FIG. 3C, the sheath support 124 may be retracted into the sheath 122 so that the shaped distal end 130 of the sheath support 124 at least partially retracts into the distal tip 126 of the sheath 122. In some embodiments, the sheath 122 can include a chamfer or flared edge near its distal end to assist in retraction of the sheath support 124 therein. In some instances, the chamfer comprises radiusing of the inside of the sheath 122 near the distal end by, for example, approximately 0.002 inches or more. Such radiusing provides an atraumatic, smooth edge to funnel the sheath support 124 into the sheath 122. Likewise, a flared edge assists in allowing the sheath support 124 to pass into the sheath 122 without hooking on the distal end of the sheath 122. This reduces any risk of damage to the sheath support 124 and reduces procedure time since the physician can reposition the device without removing the entire system.

Such retraction may be useful when it is desired to remove the shaped distal end 130 from covering the distal tip 126 of the sheath 122. Such retraction may also be used when removing the sheath support 124. By retracting the sheath support 124, the sheath support 124 can be fully withdrawn from the proximal end of the sheath 122 leaving the sheath 122 in position within the body. Thus, the sheath 122 can act as a conduit to the target, wherein the placement is undisturbed by removal of the sheath support 124 used to carefully position it. Other devices, such as leads, stylets, or other tools may then be advanced through the sheath 122 to the target. It may also be appreciated that a sheath support 124 may also be advanced through the sheath 122, such as for repositioning of the sheath 122.

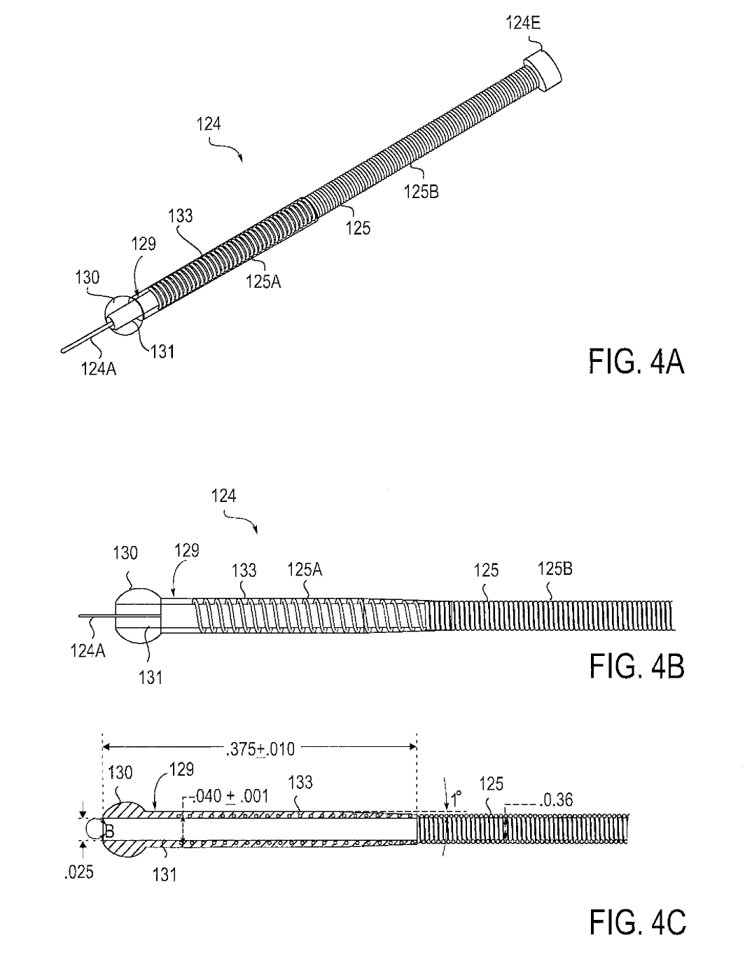

FIGS. 4A-4C illustrate an embodiment of a sheath support 124 of the present invention. In this embodiment, the sheath support 124 comprises a central shaft 125 and a distal end cap 129. The distal end cap 129 provides the rounded atraumatic tip 130 and secures the tip 130 to the shaft 125 in a manner which reduces any possibility of breakage or loss of the tip 130 from the shaft 125. Referring to FIG. 4A, the central shaft 125 comprises a coil wire which provides resiliency and flexibility. In some embodiments, the coil wire is comprised of a wire having a diameter of 0.002-0.020 inches, such 0.004 inches, and in some embodiments the central shaft 125 has an average diameter of 0.005-0.050 inches, such as 0.032 inches. Referring to FIG. 4B, in this embodiment the central shaft 125 has a distal portion 125A wherein the pitch or spacing between coil turns differs from a proximal portion 125B. In particular, in this embodiment, the pitch is greater (coil is less tightly wound) in the distal portion 125A than in the distal portion 125B. For example, the distal portion 125A may have a pitch of 0.012 inches per revolution while the proximal portion 125B has a pitch of 0.006 inches per revolution. The difference in pitch may be useful for a variety of reasons. For example, the differences in pitches can provide different flexibilities along the shaft 125. In this embodiment, the higher flexibility of the distal portion 125A assists in allowing the sheath support 124 to conform to the curvatures of the sheath 122 when it is passed therethrough. In some instances, the difference in pitch facilitates adhesion of the distal end cap 129 to the shaft 125. In some embodiments, the distal end cap 129 is attached to the shaft 125 with adhesive, wherein the larger spacing between coil turns allows for increased adhesive contact. Likewise, in some embodiments, the distal end cap 129 is attached to the shaft 125 by molding wherein the larger spacing between coil turns allows for increased molding contact. As mentioned, the distal end cap 129 provides the rounded, atraumatic distal tip 130 and secures it to the shaft 125. In some embodiments, the distal end cap 129 comprises a soft, elastomeric polymer such as silicone. Such a material allows the distal end cap 129, and particularly the rounded distal tip 130, to be retracted into the sheath 122. In addition, a variety of tip 130 designs are provided to assist in the retraction of the tip 130 into the sheath 122.

FIGS. 4B-4C provide additional views of the distal end cap 129 of FIG. 4A. In this embodiment, the end cap 129 comprises an inner tubular shaft 131, an outer tubular shaft 133 and a distal tip 130. The inner and outer tubular shafts 131, 133 are concentrically positioned and joined at least at one end by the distal tip 130. In this embodiment, the inner tubular shaft 131 extends through the distal tip 130 so that the lumen formed by the inner tubular shaft 131 is accessible via the tip 130. FIG. 4C provides a cross-sectional illustration of this embodiment of the sheath support 124. As shown, the distal end cap 129 is fitted over the distal end of the sheath support 124 so that the shaft 125 and end cap 129 share a longitudinal axis 124A and the shaft 125 is positioned between the inner and outer tubular shafts 131, 133 of the distal tip 130. In this embodiment, the inner tubular shaft 131 is tapered to assist in fitting within the shaft 125 of the sheath support 124. Mating the sheath support 124 with the end cap 129 in this manner maximizes the size of the mated surface areas between the sheath support 124 and end cap 129 which increases adhesion. Thus, the end cap 129 is fixedly attached to the sheath support 124.

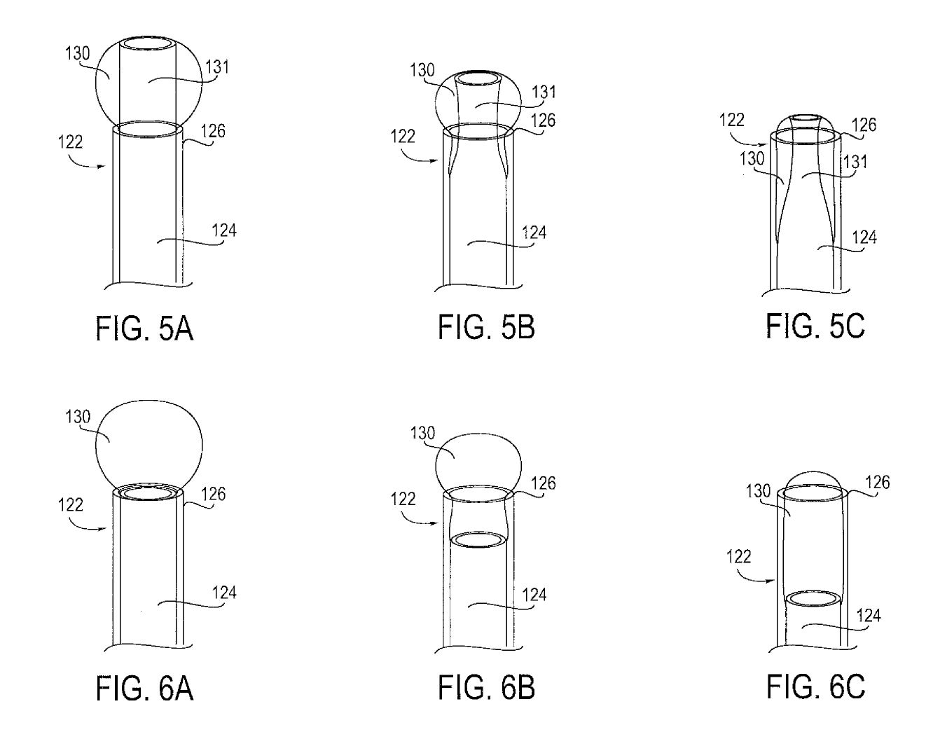

FIGS. 5A-5C illustrate the embodiment of the sheath support 124 of FIGS. 4A-4C being retracted into the sheath 122. The sheath 122 is advanceable over the sheath support 124 until its distal tip 126 abuts the rounded distal tip 130 of the sheath support 124, as shown in FIG. 5A. The distal tip 130 is rounded, having a greater diameter than the distal tip 126 of the sheath 122 so that retraction of the sheath support 124 is restricted. However, beyond a threshold force the atraumatic distal tip 130 collapses so that it can be retracted into the sheath 122. FIG. 5B illustrates the sheath support 124 being pulled into the sheath 122 with sufficient force to begin collapse of the distal tip 130 of the sheath support 124. In this embodiment, the walls of the sheath 122 apply pressure to the distal tip 130 of the sheath support 124 which in turn applies pressure to the inner tubular shaft 131 within the distal tip 130. Since the inner tubular shaft 131 is comprised of a flexible material, the inner tubular shaft 131 collapses or reduces diameter within the distal tip 130. This provides more room for the round or bulbous distal tip 130 within the sheath 122. FIG. 5C illustrates the distal tip 130 of the sheath support 124 further retracted into the sheath 122. Thus, the inner tubular shaft 131 is further collapsed therein.

FIGS. 6A-6C illustrates an embodiment of the sheath support 124 having a distal tip 130 which is comprised of a compliant solid material, such as an elastomeric polymer such as silicone, without a lumen therethrough. The sheath 122 is advanceable over the sheath support 124 until its distal tip 126 abuts the rounded distal tip 130 of the sheath support 124, as shown in FIG. 6A. Again, the distal tip 130 is rounded, having a greater diameter than the distal tip 126 of the sheath 122 so that retraction of the sheath support 124 is restricted. However, beyond a threshold force the atraumatic distal tip 130 collapses so that it can be retracted into the sheath 122. FIG. 6B illustrates the sheath support 124 being pulled into the sheath 122 with sufficient force to begin collapse of the distal tip 130 of the sheath support 124. In this embodiment, the walls of the sheath 122 apply pressure to the distal tip 130 which causes the distal tip 130 to stretch and reconfigure (changing shape) while at least partially retracting into the lumen 123 of the sheath 122 due to the flexible and compliant nature of the solid material. FIG. 6C illustrates the distal tip 130 of the sheath support 124 further retracted into the sheath 122.

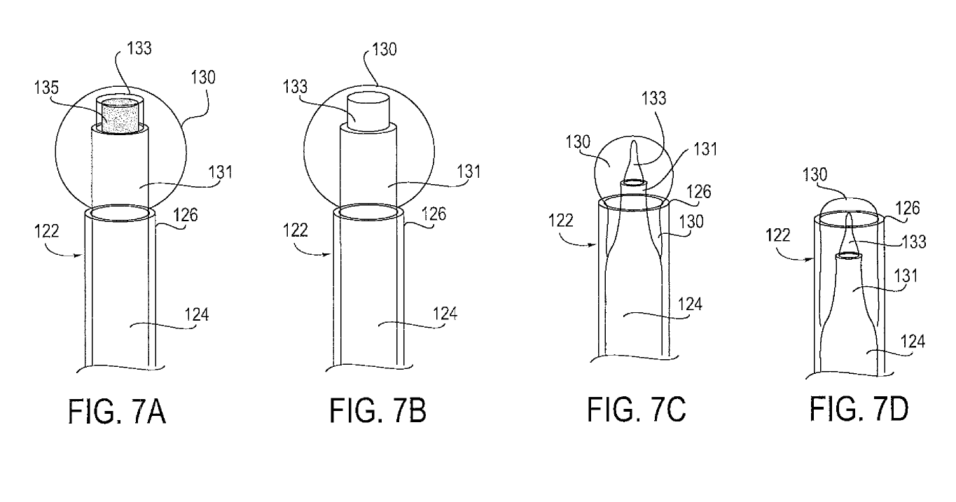

FIGS. 7A-7D illustrate another embodiment of a distal tip 130 of a sheath support 124. In this embodiment, the inner tubular shaft 131 does not extend through the tip 130. Rather, the inner tubular shaft 131 extends partially within the rounded distal tip 130. Distal to the inner tubular shaft 131 resides a cavity, space or gap 133. The gap 133 aligns with the inner tubular shaft 131 so that a rod or stylet 135 is advanceable within the inner tubular shaft 131 and into the gap 133, as illustrated in FIG. 7A. Positioning the stylet 135 within the gap 133 holds the gap 133 open and supports the distal tip 130 of the sheath support 124. Thus, the sheath support 124 may be delivered with the stylet 135 in place. The sheath support 124 is positionable within the sheath 122 so that its distal tip 126 abuts the rounded distal tip 130 of the sheath support 124. When it is desired to retract the distal tip 130 within the sheath 122, the stylet 135 is retracted into the inner tubular shaft 131 revealing the gap 133 within the distal tip 130, as illustrated in FIG. 7B. Optionally, the stylet 135 may be completely removed. The sheath support 124 is then retracted into the sheath 122 by applying a threshold force longitudinally along the axis 124A. The atraumatic distal tip 130 collapses so that it can be retracted into the sheath 122. FIG. 7C illustrates the sheath support 124 being pulled into the sheath 122 with sufficient force to begin collapse of the distal tip 130 of the sheath support 124. In this embodiment, the walls of the sheath 122 apply pressure to the distal tip 130 of the sheath support 124 which in turn applies pressure to the inner tubular shaft 131 within the distal tip 130. Since the inner tubular shaft 131 is comprised of a flexible material, the inner tubular shaft 131 collapses or reduces diameter within the distal tip 130. Likewise, the gap 133 collapses, further assisting compression of the distal tip 130. This provides more room for the round or bulbous distal tip 130 within the sheath 122. FIG. 7D illustrates the distal tip 130 of the sheath support 124 further retracted into the sheath 122. Thus, the inner tubular shaft 131 and gap 133 are further collapsed therein.

It may be appreciated that in other embodiments, the sheath support 124 is constructed similarly to FIGS. 7A-7D however the inner tubular shaft 131 does not extend into or as far into the distal tip 130. For example, in some embodiments the inner tubular shaft 131 begins proximal to the rounded distal tip 130 and the gap 133 extends within the distal tip 130. In this embodiment, when the tip 130 collapses, the gap 133 collapses within. It may also be appreciated that more than one gap 133 may be present and each gap 133 may be of various sizes and shapes. Likewise, each gap 133 may extend various lengths within the distal tip 130. In some embodiments, one or more gaps 133 are not aligned with the longitudinal axis 124A. In some embodiments, the distal tip 130 is simply hollow wherein the gap 133 forms the hollow portion within the distal tip 130. In such hollow embodiments, the outer surface of the distal tip 130 may be continuous or have openings to the hollow interior. Likewise, in any of the embodiments, the outer surface of the distal tip 130 may be continuous or have openings to one or more interior gaps 133. It may also be appreciated that in some embodiments the distal tip 130 does not include any gaps 133, such as illustrated in FIGS. 6A-6C. In any of these embodiments, the surface of the distal tip 130 may have openings, such as cuts, slices or holes, which do not connect with an inner gap or the lumen of the inner tubular shaft 131. Each of these configurations may assist in collapse of the distal tip 130 for retraction into the sheath 122.

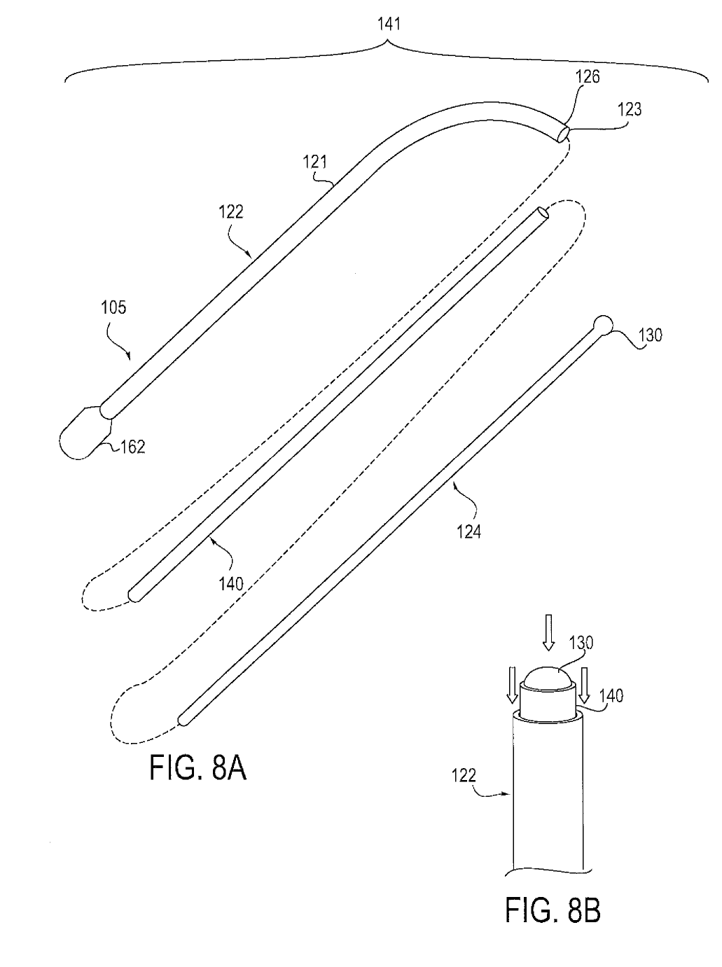

In some embodiments, a retraction shield 140 is used to assist in retracting the sheath support 124. Typically, the retraction shield 140 has the form of an elongate tubular shaft 142 having a lumen therethrough 144. FIGS. 8A-8B illustrate an embodiment of a system 141 including a sheath 122, a retraction shield 140 and sheath support 124. As shown in FIG. 8A, the sheath 122 has a lumen 123 therethrough and a retraction shield 140 is positionable within the lumen 123 of the sheath 122. Likewise, the sheath support 124 is positionable within the lumen 144 of the retraction shield 140. The system 141 is advanceable within the epidural space. Together, the sheath 122, shield 140, and sheath support 124 provide desirable flexibility and steering capabilities within the epidural space while resisting kinking. Once the system 141 is desirably positioned, the retraction shield 140 and sheath support 124 may be withdrawn by retracting the sheath support 124 so that the distal tip 130 is compressed within the retraction shield 140 (as illustrated in FIG. 8B) and then pulling the retraction shield 140 proximally out of the sheath 122. This allows the distal tip 130 to simply wedge within the retraction shield 140 and the retraction shield 140 is easily withdrawn without friction within the sheath 122. This may increase speed of removal and ease of use.

The above described devices can be used for delivery of a lead or various tools to a target location within the body, particularly via the epidural space, and more particularly to a target dorsal root ganglion. Thus, embodiments of epidural delivery methods of the present invention are described herein. In particular, such embodiments are described and illustrated as an antegrade approach. It may be appreciated that, alternatively, the devices and systems of the present invention may be used with a retrograde approach or a contralateral approach. Likewise, at least some of the devices and systems may be used with a transforaminal approach, wherein the DRG is approached from outside of the spinal column. Further, the target DRG may be approached through the sacral hiatus or through a bony structure such as a pedicle, lamina, or other structure.

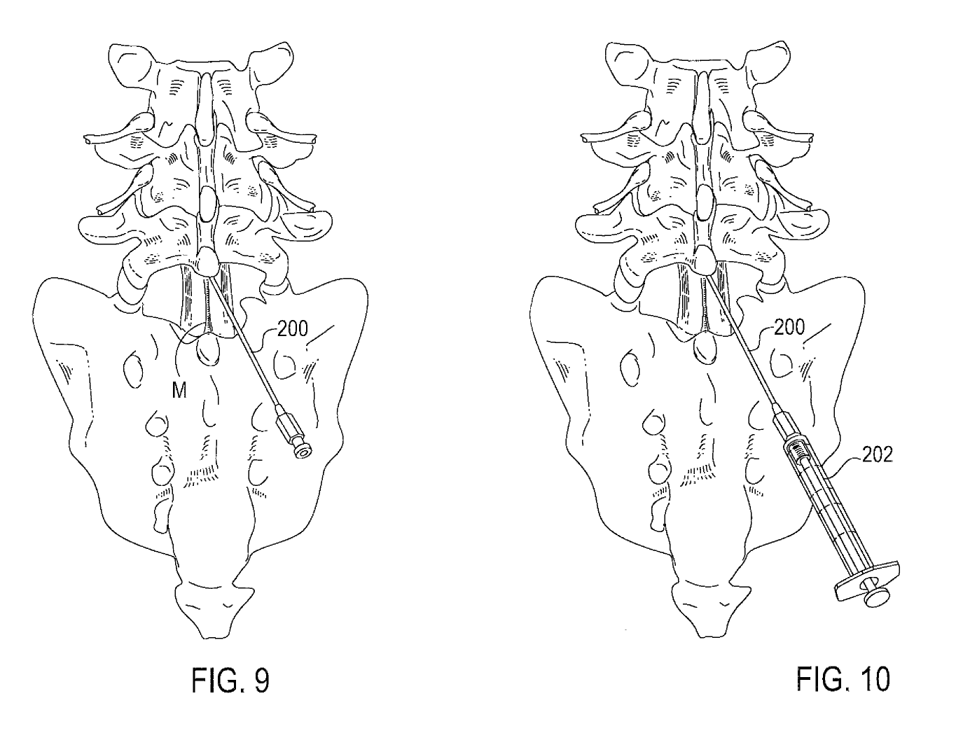

Epidural delivery involves accessing the epidural space. The epidural space can be accessed with the use of an introducing needle 200, as illustrated in FIG. 9. The insertion point is usually near the midline M, although other approaches may be employed. Typically, the needle 200 is inserted through the ligamentum flavum and a loss of resistance to injection technique is used to identify the epidural space. Referring to FIG. 10, a syringe 202 is attached to the needle 200. Once the tip of the needle 200 has entered a space of negative or neutral pressure (such as the epidural space) and a “loss of resistance” is felt, it will be possible to inject through the syringe 202. In addition to the loss of resistance technique, real-time observation of the advancing needle 200 may be achieved with a portable ultrasound scanner or with fluoroscopy. Likewise, a device may be advanced through the needle 200 and observed within the epidural space with the use of fluoroscopy.

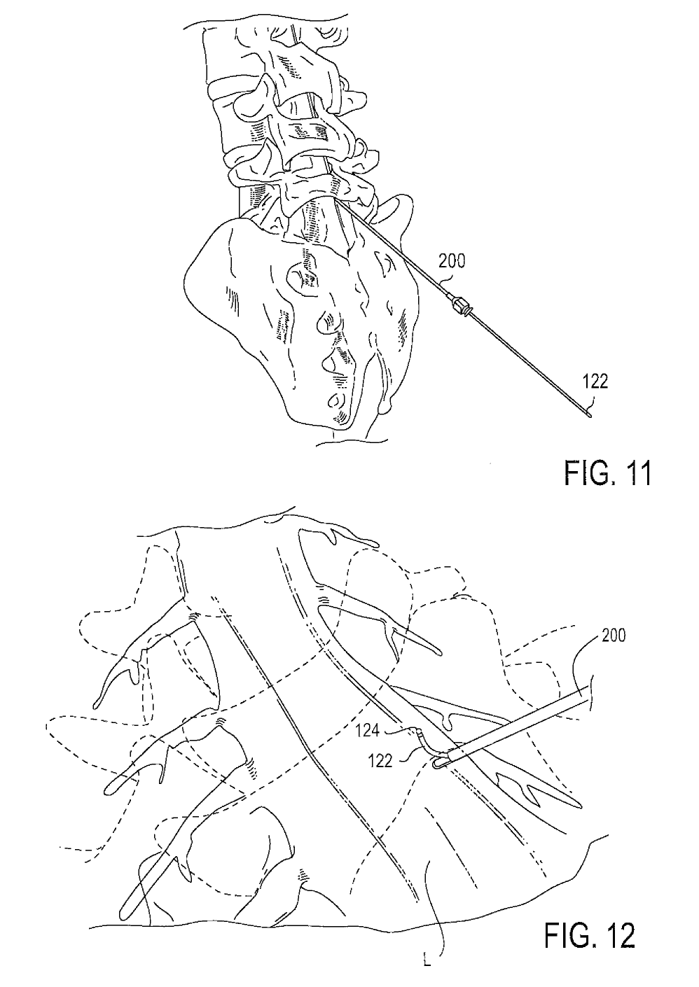

Once the needle 200 has been successfully inserted into the epidural space, the syringe 202 can be removed. The sheath support 124 is either preloaded in the sheath 122 or the sheath support 124 may be positioned within the sheath 122, preferably by advancing the sheath 122 over the sheath support 124 or optionally by advancing the sheath support 124 through the sheath 122. Typically, the sheath 122 is positioned so that the distal tip 126 of the sheath 122 abuts the rounded ball shape of the distal tip 130 of the sheath support 124. In this position, the sheath support 124 conforms to the pre-curved shape of the sheath 122. The sheath support 124 and the sheath 122 are inserted through the needle 200, into the epidural space, as illustrated in FIG. 11.

Referring to FIG. 12, the distal end of the needle 200 is shown passed through the ligamentum flavum L and the assembled sheath 122 and sheath support 124 is shown emerging therefrom. The rigidity of the needle 200 straightens the more flexible sheath 122 and sheath support 124 as they pass therethrough. However, upon emergence, the sheath 122 is allowed to bend along or toward its precurvature as shown. Such bending assists in steering within the epidural space. This can be particularly useful when using a retrograde approach to navigate across the transition from the lumbar spine to the sacral spine. The sacrum can create a “shelf” that can resists ease of passage into the sacrum. The precurved sheath 122 is able to more easily pass into the sacrum, reducing operating time and patient discomfort. As discussed above, the sheath support 124 comprises an atraumatic distal tip 130 that minimizes the risk of injury to the patient as the assembled sheath 122 and sheath support 124 is advanced into the sacrum, the epidural space, and other anatomies.

Referring to FIG. 12, the assembled sheath 122 and sheath support 124 is advanced within the epidural space toward a target DRG. Steering and manipulation can be controlled proximally and is assisted by the construction of the assembled components. In particular, the sheath 122 and sheath support 124 assembly is designed to reduce or eliminate kinking of the sheath 122. In some instances, advancement of the sheath 122 alone or with a device therein, such as a lead, can allow kinks to form in the sheath 122. This can occur during introduction into the needle (due to mishandling of the distal tip of the sheath) or during deployment of the sheath into the epidural space (due to the distal end of the sheath encountering bone or other tissue that causes the sheath to undergo significant bending. Once the sheath is kinked, deployment of the lead (or other device) therein is hindered. In some instances, such hindering completely obstructs deployment. This is because the sheath kink reduces the inner diameter of the sheath, pinching the lead or device in place. In addition, such kinking can make removal of the sheath through the needle difficult. In such instances, the sheath kink has increased the outer diameter of the sheath, binding the sheath in the needle. Since the sheath support 124 has an outer diameter that closely fits within the lumen 123 of the sheath 122 while maintaining free sliding therein, the sheath support 124 maintains the inner diameter of the lumen 123 of the sheath 122 and prevents the sheath walls from collapsing into a kink, particularly during low radius bends that may occur during delivery within the body. Thus, such a design is particularly useful during insertion through the needle 200 and advancement within the epidural space, particularly when approaching a target DRG such as when advancing laterally outward, away from the midline of the spinal column, along a dorsal root.

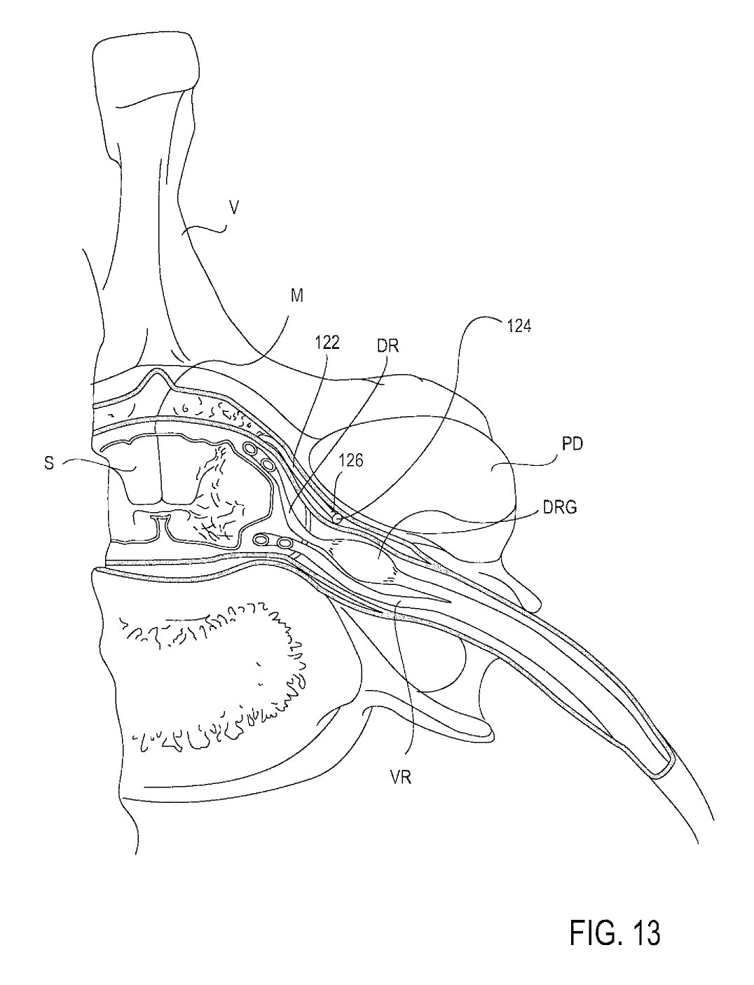

FIG. 13 illustrates a cross-sectional view of a vertebrae V and spinal column S, including the sheath 122 and sheath support 124 (of FIG. 12) directed laterally outward, away from the midline of the spinal column M, along a dorsal root DR. The sheath 122 is advanceable up to the foramen, at least partially within the foramen, within the foramen, or through the foramen, for delivery of a lead or devices to a desired location. In many instances, the area around the DRG, particularly within the foramen, is restricted or tight. The foramen is a confined area by nature and in some patients stenosis occurs in the foramen further restricting the area. Foraminal stenosis may be caused by a congenital condition and some people are genetically predisposed to this condition, however it is more often brought on through the natural aging process or various disease states.

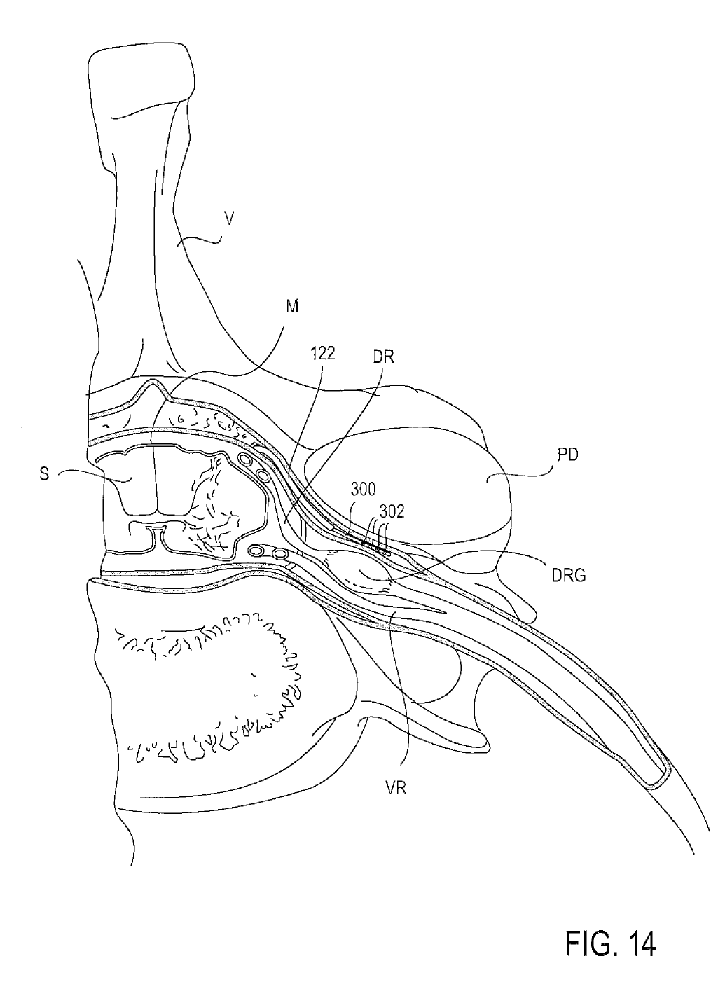

FIG. 13 illustrates the sheath 122 and sheath support 124 assembly advanced at least partially within the foramen, the distal tip 130 of the sheath support 124 abutting the distal tip 126 of the sheath 122. Such positioning of the sheath 122 may be sufficient for delivery of a lead or devices through the sheath 122 to the DRG. Thus, the sheath support 124 may be removed by retraction, collapsing the rounded distal tip 130 within the sheath 122 and withdrawing the sheath support 124 proximally, leaving the sheath 122 in place. This provides a conduit to the DRG for delivery of a lead or other devices thereto. FIG. 14 illustrates a lead 300 advanced through the previously positioned sheath 122 and positioned so that at least one electrode 302 is in proximity to the DRG, in a manner so as to allow selective stimulation of the DRG.

As used herein in at least one embodiment, selective stimulation of the DRG means that the stimulation substantially only neuromodulates or neurostimulates a dorsal root ganglion. In some embodiments, selective stimulation of a dorsal root ganglion leaves the motor nerves or the ventral root VR unstimulated or unmodulated. In some embodiments, selective stimulation means that within the nerve sheath, the A-myelinated fibers are preferentially stimulated or neuromodulated as compared to the c-unmyelinated fibers. As such, embodiments of the present invention advantageously utilize the fact that A-fibers carry neural impulses more rapidly (almost twice as fast) as c-fibers. Some embodiments of the present invention are adapted to provide stimulation levels intended to preferentially stimulate A-fibers over c-fibers.

When positioned for selective stimulation, at least one electrode is in close proximity to the target, such as on, about, near, against, adjacent to the target. When stimulating the dorsal root ganglion, the at least one electrode is typically at least partially within the foramen. Often the at least one electrode is against or in contact with the outer dura layer around the DRG. In some embodiments, the at least one electrode is position within the dura layer, such as within the DRG. However, in other embodiments, the at least one electrode is near, adjacent or in close proximity to the DRG. In any case, the position of the at least one electrode in combination with the electrical signal provided to the at least one electrode selectively stimulates the DRG while avoiding or providing little or imperceptible amounts of stimulation energy to tissues undesired for stimulation, such as the nearby ventral nerve root.

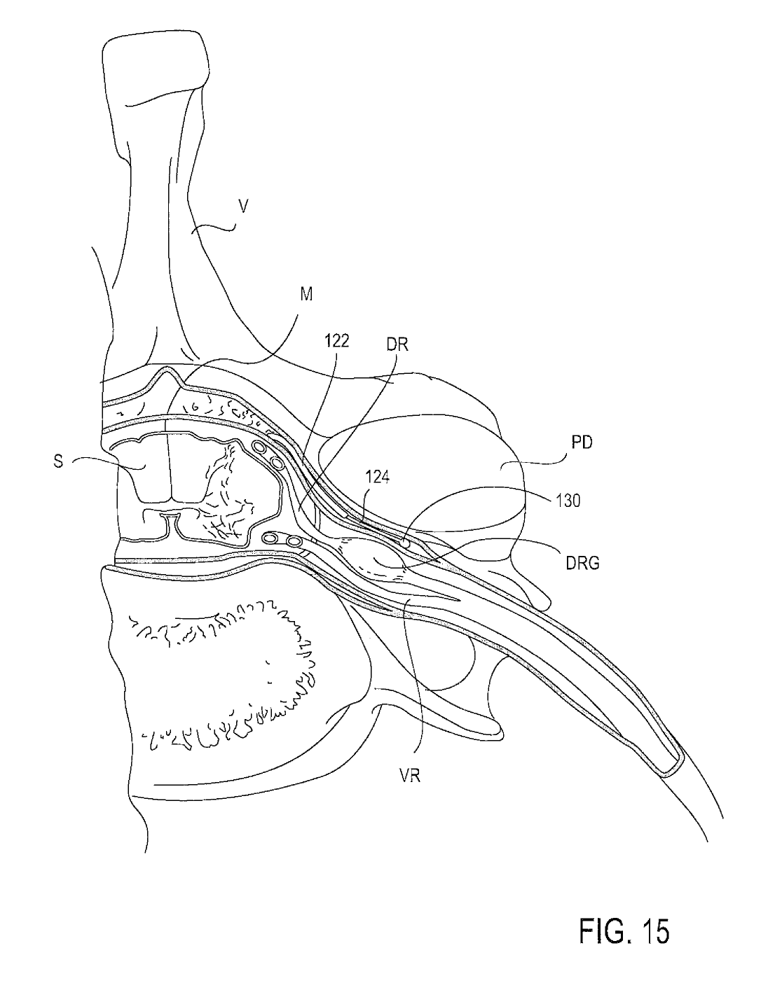

In some embodiments, the lead 300 is not easily advanceable beyond the sheath 122, such as due to foraminal stenosis, connective tissue, fascia and/or scar tissue. In such instances, prior to removal of the sheath support 124, the sheath support 124 may be advanced distally, as illustrated in FIG. 15, so that the distal tip 130 is located a distance away from the distal tip 126 of the sheath 122. Since the sheath support 124 has desirable rigidity and steerability, the sheath support 124 may be advanced through resistant areas, such as in the manner of a tunneling tool. The atraumatic distal tip 130 protects the tissue in the area from damage or puncture, creating a pathway for a lead or other device. FIG. 15 illustrates the sheath support 124 being advanced further into the foramen, adjacent the dorsal root ganglion.

It may be appreciated that in some instances such tunneling may be treatment for the patient in and of itself. For example, when a foramen is restricted, compression of the nerve root inside produces a nerve injury called a radiculopathy. In addition to pain, a radiculopathy can trigger various changes in normal nerve function. Depending on the location of the stenosis, these changes can manifest in the arms or legs as burning or tingling sensations and muscle weakness. In some instances, tunneling within the foramen with a device, such as the sheath support 124, can create additional space within the foramen sufficient to reduce compression of the nerve.

It may be appreciated that in some embodiments, the distal tip 130 has a shape differing from the atraumatic rounded shape described above. For example, the distal tip 130 may have a pointed end, a cutting edge or a shape configured for penetrating or removing tissue, such as tissue causing the foraminal stenosis. Alternatively, the sheath support 124 may be withdrawn and a tool may be inserted through the sheath 122 for performing a particular procedure. Examples of such tools include cutters, clippers, shavers, grinders, etc. Likewise, a vacuum tool may be inserted to remove material from the area. Once the procedure(s) have been completed, the condition may be adequately treated wherein the sheath 122 is removed. Alternatively, a lead 300 may be advanced through the sheath 122, such as illustrated in FIG. 14, within the tunnel or newly expanded anatomy for neuromodulation of the DRG.

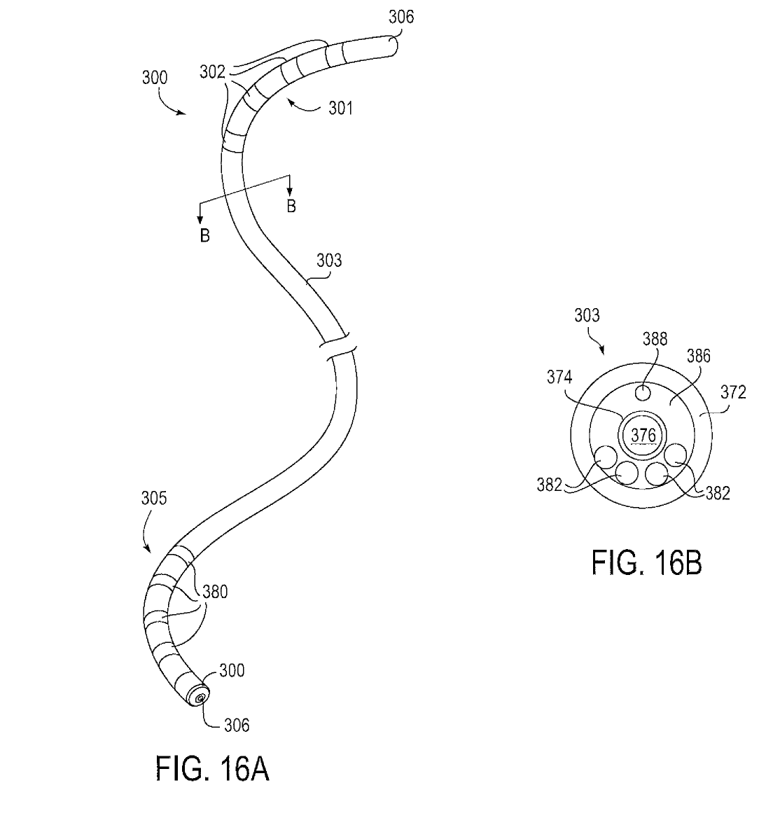

The lead 300 may have a variety of forms. FIGS. 16A-16B illustrate one embodiment of such a lead 300. Referring to FIG. 16A, in this embodiment the lead 300 comprises a shaft 303 having a distal end 301 and a proximal end 305. At least one electrode 302 is disposed along the distal end 301. It may be appreciated that any number of electrodes 102 may be present, including one, two, three, four, five, six, seven, eight or more. In the embodiment if FIG. 16A, the lead 300 includes four electrodes 302 disposed along its distal end 301. Typically, the electrodes 302 are comprised of platinum or platinum/iridium alloy. In this embodiment, the electrodes 302 have a ring shape, extending around the shaft 303, and have an outer diameter approximately equal to the outer diameter of the shaft 303. In some embodiments, the electrodes have a wall thickness of approximately 0.002-0.004 inches and a length of approximately 0.030-0.060 inches or greater. It may be appreciated that the shaped distal tip 306 of the lead 100 may be formed from the most distal electrode. In this embodiment, the distal end 301 has a closed-end distal tip 306. The distal tip 306 may have a variety of shapes including a low profile rounded shape as shown. Typically the distal tip 306 has a profile low enough to pass through the sheath 122 with minimal or no friction. In some embodiments, the lead 300 also includes a stylet lumen which extends toward the closed-end distal tip 306, however such a lumen is optional.

It may also be appreciated that the electrodes 302 may have other forms. For example, in some embodiments, at least one electrode 302 may be comprised of a plurality of elements that are electrically connected to each other. In other embodiments, at least one electrode 302 extends partially around the shaft of the lead 300 so as to impart a directional field. In still other embodiments, at least one electrode may have a hollow cylinder shape wherein one or more features are cut from or through its surface. This may allow extension of the length of the electrode without increasing its surface area. Such longer electrodes may reduce the effects of lead migration. Other embodiments include diverse electrode shapes and edge geometries in order to affect the level and variation of current density to optimize the effect of the energy on the target anatomy. It may also be appreciated that at least one electrode 302 may have a composite structure or be comprised of pyrolite carbon which provides for surface geometry increases.

The lead 300 also includes at least one electrical contact 380 disposed near its proximal end 305 which is removably connectable with a power source, such as an implantable pulse generator IPG. In this embodiment, the lead 300 includes a corresponding electrical contact 380 for each electrode 302. Electrical energy is transmitted from the electrical contact 380 to the corresponding electrode 302 by a conductor cable 382 which extends therebetween. Thus, the cables 382 are typically approximately 18-22 inches long, but are typically up to 120 cm (47.24 inches) long.

FIG. 16B provides a cross-sectional view of the shaft 303 of FIG. 16A. The shaft 303 comprises a single lumen tube 372 formed from an extruded polymer, such as urethane. The lead 300 will typically have similar dimensions, particularly cross-sectional dimensions, to the sheath support 124 described above so that the sheath support 124 can be easily exchanged for the lead 300 within the sheath 122 as described above. Typically, the tube 372 has an outer diameter in the range of approximately 0.040-0.050 inches, a wall thickness in the range of approximately 0.005-0.010 inches and a length of approximately 12-30 inches. However, such dimensions serve only as an example. For instance, in other embodiments, the tube 372 may have an outer diameter in the range of approximately 0.028-0.050 inches, a wall thickness in the range of approximately 0.003-0.010 inches and a length of approximately 30-120 cm. It may be appreciated that other materials may be used, such as silicone or other commonly used implantable polymers.

Referring to FIG. 16B, the lead 300 may also include a stylet tube 374 disposed within the single lumen tube 372. The stylet tube 374 can form a stylet lumen 376 and isolates an optional stiffening stylet from the other components of the lead 300. The stylet tube 374 can also provide a smooth or lubricious surface against which the stylet 324 passes during insertion and retraction. Such lubriciousness may be desirable to resist jamming or hang-ups of a highly curved stiffening stylet within the lead 300. In addition, the lubricious surface reduces the effects on delivery of contamination by bodily fluids. The stylet tube 374 may also provide tensile strength to the lead 300 during delivery. In many embodiments, the use of the stiffening stylet may not be necessary and the lead 300 may not necessarily need to include the stylet 374.

In some embodiments, the stylet tube 374 is comprised of polyimide. Polyimide is a biocompatible, high strength, smooth, flexible material. Smoothness can be provided by the means of manufacturing, and adequate lubriciousness may be provided by the low coefficient of friction (0.7) of the material. In some embodiments, the polyimide is combined with Teflon to lower the coefficient of friction while maintaining high strength. Because polyimide is high strength, tough and smooth, stiffening stylets having highly radiused bends may be easier to introduce and manipulate therein without the stiffening stylets catching, hanging, jamming, or piercing into or through the sides of the stylet tube 374 as may occur with some polymers. In some embodiments, the polyimide material can be loaded with a strengthening material to increase its overall tensile strength. Examples of such strengthening materials include engineering fibers, such as Spectra® fiber, Vectran™ fiber, and Kevlar™ fiber, to name a few.

The physical qualities of the polyimide material can also allow the stylet lumen walls to be very thin, such as approximately 0.001 inches or less, which can help to minimize the overall diameter of the lead 300. Such thinness may not be achieved with the use of some other biocompatible polymer materials with equivalent strength and resistance to buckling.

In other embodiments, the stylet tube 374 is comprised of polyetheretherketone (PEEK). PEEK is a biocompatible, high strength, and smooth material, and in a thin-walled tube configuration is a sufficiently flexible material. Smoothness can be provided by the means of manufacturing, and adequate lubriciousness can be provided by the fairly low coefficient of friction (0.35) of the material. Because PEEK is high strength, tough and smooth, stiffening stylets having highly radiused bends may be easier to introduce and manipulate therein without the stiffening stylet catching, hanging, jamming or piercing into or through the sides of the stylet tube 374 as may occur with some polymers.

And, in other embodiments, the stylet tube 374 may be comprised of other polymers, such as Polyethylene Terephthalate (PET) film (also known as polyester or Mylar), or other materials, such as a metal tube, a flexible metal tube (such as formed from nitinol), a laser-cut metal tube, a spring or coil (such as a metal close-coiled spring), or a combination of materials and forms.

As mentioned above, the stylet tube 374 may have a lubricious surface, such as a coating or embedded layer, along at least a portion of the stylet lumen 376 to provide the desired lubriciousness. An example of such a surface is a polytetrafluoroethylene (PTFE) or parylene coating. The tube 374 may be comprised of a material such as polyimide and additionally coated, or the tube 374 may be comprised of a less lubricious material and coated to attain the desired lubricity. Such a coating may be particularly useful when the shaft 303 is comprised of a multi-lumen extrusion.

It may be appreciated that alternatively, a multi-lumen tube may be used for the shaft 303 of the lead 300, or a combination of multi-lumen and single lumen tubing. When such a multi-lumen tube is formed from an extruded polymer, various other components of the lead 300 may be coextruded with the multi-lumen tube (such as conductor cables, a stylet tube and/or a tensile wire described herein below). In one embodiment, the shaft 303 of the lead 300 is a five lumen extrusion. Four of the lumens house conductor cables; each conductor cable loosely filling each lumen. And, one larger lumen serves as the stylet lumen 176. Typically, the stylet lumen 176 includes a lubricious surface 175, such as a coating or embedded layer, along at least a portion of the stylet lumen 176 to provide the desired lubriciousness. In addition a tensile element 188 may be co-extruded with the extrusion or the tensile element may be loosely embedded in a sixth lumen of the extrusion. The ability to per-insert a cable or element loosely into a small lumen is a specialized aspect that allows the lead 300 increased flexibility. And, although the lead 300 may be typically curved by devices such as a stylet, the distal end of the multi-lumen tube may optionally be thermally precurved to assist in such curvatures.

Referring again to FIG. 16B, in this embodiment the conductor cables 382 extend through a space 186 between the stylet tube 374 and the single lumen tube 372. The cables 382 may be comprised of any suitable material, preferably multiple Drawn Filled Tube (DFT) strands each comprising a high strength outer layer of cobalt-chrome alloy and a high conductivity core of silver, platinum or platinum/iridium alloy. Typically, the cables 382 are electrically insulated by a thin layer of material, such as polytetrafluoroethylene (PTFE) or perfluoroalkoxy (PFA). Consequently, the cables 382 typically have an outer diameter of approximately 0.006 inches. However, it may be appreciated that the cables 382 may be uncoated or uninsulated when the shaft 303 is comprised of a multi-lumen extruded tube and each cable 382 extends through a dedicated lumen, or alternatively, when the cables are embedded in the wall of the extruded tube. Another type of cable construction can include a combination of high strength strands and high conductivity strands. Alternatively, only high strength strands, such as cobalt-chrome alloy or stainless steel, may be used. In such embodiments, resistance may be decreased by enlarging the cable cross section.

Each cable 382 can be joined to an electrode 302 and a corresponding electrical contact 380 by a suitable method, such as welding, brazing, soldering or crimping, to name a few. The joining process can provide an electrical contact between the cable and the electrode, and also resists separation of the cable from the electrode due to any tensile forces that the lead may be subjected to during or after implantation. Therefore, the joining process should be electrically low resistance and be physically high strength. A high strength joint can be enabled by ensuring that neither of the materials being joined is degraded by the joining process, in addition to having sufficient surface area, compatible materials and other factors. In preferred embodiments, such joining can be achieved by welding which is performed using a YAG laser from the outside of the electrode 302, through the electrode wall. The laser joins the cable 382 with the inner surface of the electrode 302. In some embodiments, the weld melts the electrode alloy so that the melt at least partially penetrates the strands of the cable 382 which are touching the inner surface of the electrode 302. It is desirable that little melting of the cable 382 (e.g. strands of DFT) occurs because the strength properties of cobalt-chrome alloy may decrease when it is overheated due to welding.

In some embodiments, each electrode 302 is welded to the conductor cable 382 with two welds. The two welds can be approximately 0.020-0.040 inches apart along the electrode 302. When stranded cables are used, twisting of the strands between the two welds can capture a different set of strands in each weld. After the welding is complete, the strands at the end of the cable 382 may be laser fused together by cutting the cable 382 to length near the end of the electrode 302. It may be appreciated that the same methods may be used to weld the cable 382 to the corresponding electrical contact 380.

This welding method can ensure that many strands are captured by the welds to connect the cable 382 with the electrode 302 or electrical contact 380 without overheating the cable material. However, it may be appreciated that a single weld may be used. In any case, fusing the end of the cable 382 after welding can increase the load sharing of the strands and the breaking strength of the cable weld. Thus, even those strands that are not directly welded to the electrode 302 or electrical contact 380 can at least partially share the tensile load through the fusing operation.

It may be appreciated that, in some embodiments, at least some of the cables 382 are comprised of a single wire. In such instances, a single weld may be sufficient. In other embodiments, the cables 382 are formed together in a composite cable. Optionally, the cables 382 may be embedded in the wall of the shaft 303.

In some embodiments, the lead 300 also includes a tensile element 388, as illustrated in FIG. 16B. The tensile element 388 can extend through the space 386 between the stylet tube 374 and the single lumen tube 372. In some embodiments, the tensile element 388 can comprise a single strand wire of suitable material, such as cobalt-chrome alloy. In such embodiments, the element 388 typically has a diameter of 0.004 inches. Optionally, the element 388 may have multiple diameters. For instance, the element 388 may have a larger diameter near the proximal end 305 (such as approximately 0.010 inches) and then neck down toward the distal end 301. This may increase the ease of insertion of at least a portion of the proximal end 305 into the implantable pulse generator yet maintain adequate flexibility in the distal end 301 of the lead 300 while retaining adequate tensile strength. It may be appreciated that in some embodiments, more than one tensile element 388 may be used. And, in some embodiments the tensile element 388 is comprised of other materials and forms such as metals, polymers, stainless steel, braids, and cables, to name a few.

The element 388 typically extends from the distal end 301 to the proximal end 305 of the lead 300; however the element 388 may extend any desirable distance. The element 388 may be fastened to portions of the lead 300 that allow the element 388 to absorb tensile stress applied to the lead 300 during or after implantation. In particular, the element 388 may be tighter or straighter than the conductor cables 382 so as to absorb the tensile load first. Thus, the tensile element 388 may be flexible, at least near the distal end 301, but can have adequate tensile strength (such as greater than or equal to 2 lbf) to guard the cables 382 and welds from breakage. This may be preferable to the conductor cables 382 and welds absorbing the tensile load and increases the tensile strength of the lead 300. Such fastening may be achieved with welding, potting, crimping, wrapping, insert molding or any suitable method.

In the embodiment of FIG. 16B, the stylet tube 374, the tensile element 388, and the conductor cables 382 can extend through the single lumen tube 372 and are free to move therein. Typically, these components may be fixed to the single lumen tube 372 near its proximal and distal ends and the components are unattached therebetween. Thus, as the lead 300 bends or curves during positioning, the stylet tube 374, the tensile element 388, and the conductor cables 382 are each able to move somewhat independently within the single lumen tube 372. Such movement can allow greater flexibility in bending and lower applied forces to achieve reduced curve radii in the lead 300. It may be appreciated that the components may be fixed at other locations, allowing freedom of movement therebetween. Likewise, it may be appreciated that the space 386 may optionally be filled with potting material, such as silicone or other material.

In some embodiments, the lead 300 does not include a separate tensile element 388. In such embodiments, the stylet tube 374 may optionally be reinforced with longitudinal wires, strips, coils, embedded braids or other elements to provide additional tensile strength. And, in some embodiments, the lead 300 does not include a tensile element 388 or stylet tube 374.

Referring back to FIG. 14, the DRG may then be stimulated by providing stimulation energy to the at least one electrode 302. It may be appreciated that multiple electrodes may be energized to stimulate the target DRG. It may also be appreciated that the electrodes may be energized prior to removal of the sheath 122, particularly to ascertain the desired positioning of the lead 100.

The same needle 200 can then be used to position additional leads within the epidural space. In some embodiments, the sheath 122 is removed and a new sheath 122/sheath support 124 assembly is advanced through the needle 200 to a new target, such as a DRG on a different spinal level or a different DRG on the same spinal level. The same methods as described above are then utilized to access the new target. In other embodiments, a sheath support 124 is advanced within the previously positioned sheath 122 and the sheath 122/sheath support 124 assembly is steered toward a new target, such as a DRG on a different spinal level or a different DRG on the same spinal level.

It may be appreciated that any number of leads 300 may be introduced through the same introducing needle 200. In some embodiments, the introducing needle 200 can have more than one lumen, such as a double-barreled needle, to allow introduction of leads 200 through separate lumens. Further, any number of introducing needles 200 may be positioned along the spinal column for desired access to the epidural space. In some embodiments, a second needle can be placed adjacent to a first needle. The second needle can be used to deliver a second lead to a spinal level adjacent to the spinal level corresponding to the first needle. In some instances, there may be a tract in the epidural space and the placement of a first lead may indicate that a second lead may be easily placed through the same tract. Thus, the second needle can be placed so that the same epidural tract may be accessed. In other embodiments, a second needle may be used to assist in stabilizing the tip of a sheath inserted through a first needle. In such embodiments, the second needle may be positioned along the spinal column near the target anatomy. As the sheath is advanced, it may use the second needle to buttress against for stability or to assist in directing the sheath. This may be particularly useful when accessing a stenosed foramen which resists access.

It may be appreciated that the sheath support 124 may include a drug or agent delivery tips or the sheath support 124 may be removed and replaced with an elongate device which delivers a drug or agent, such as a catheter or a lead. In one embodiment, the elongate device comprises a delivery element having a lumen for delivery of at least one agent to at least one target neural anatomy, such as described and illustrated in U.S. patent application Ser. No. 13/309,429 entitled “Directed Delivery of Agents to Neural Anatomy”, filed on Dec. 1, 2011, incorporated herein by reference for all purposes.

Example agents include analgesics and pain medicine. In some embodiments, an agent which is delivered can be, for example, but is not limited to, one or more or a combination of: lidocaine, epinephrine, fentanyl, fentanyl hydrochloride, ketamine, dexamethasone, hydrocortisone, peptides, proteins, Angiotension II antagonist, Antriopeptins, Bradykinin, Tissue Plasminogen activator, Neuropeptide Y, Nerve growth factor (NGF), Neurotension, Somatostatin, octreotide, Immunomodulating peptides and proteins, Bursin, Colony stimulating factor, Cyclosporine, Enkephalins, Interferon, Muramyl dipeptide, Thymopoietin, TNF, growth factors, Epidermal growth factor (EGF), Insulin-like growth factors I & II (IGF-I & II), Inter-leukin-2 (T-cell growth factor) (I1-2), Nerve growth factor (NGF), Platelet-derived growth factor (PDGF), Transforming growth factor (TGF) (Type I or δ) (TGF), Cartilage-derived growth factor, Colony-stimulating factors (CSFs), Endothelial-cell growth factors (ECGFs), Erythropoietin, Eye-derived growth factors (EDGF), Fibroblast-derived growth factor (FDGF), Fibroblast growth factors (FGFs), Glial growth factor (GGF), Osteosarcoma-derived growth factor (ODGF), Thymosin, or Transforming growth factor (Type II or β) (TGF). In some embodiments, an agent delivered is selected from one or more or a combination of: opioids, COX inhibitors, PGE2 inhibitors, Na+ channel inhibitors.

In some embodiments of all aspects of the invention as disclosed herein, an agent which is delivered can be, for example, an agonist or antagonist of a receptor or ion channel expressed by a dorsal root ganglion, for example, an agonist or antagonist of a receptor or ion channel which is upregulated in a dorsal root ganglion in response to nerve injury, inflammation, neuropathic pain, and/or nociceptive pain. In some embodiments, an ion channel expressed by the dorsal root ganglion is selected from any one of, or a combination of: voltage gated sodium channels (VGSC), voltage gated Calcium Channels (VGCC), voltage gated potassium channel (VGPC), acid-sensing ion channels (ASICs). In some embodiments, a voltage-gated sodium channel (VGSC) includes TTX-resistant (TTX-R) voltage gated sodium channels, such as, but not limited to, Nav1.8 and Nav1.9. In some embodiments, a voltage-gated sodium channel (VGSC) is a TTX-sensitive (TTX-S) voltage gated sodium channel, for example, but not limited to, Brain III (Nav1.3). In some embodiments, a receptor is selected from any one of, or a combination of, ATP receptor, NMDA receptors, EP4 recetors, metrix metalloproteins (MMPs), TRP receptors, neurtensin receptors.

Further example agents and methods of use are also described and illustrated in U.S. patent application Ser. No. 13/309,429 entitled “Directed Delivery of Agents to Neural Anatomy”, filed on Dec. 1, 2011, incorporated herein by reference for all purposes.

It may be appreciated that the devices, systems and methods of the present invention may be used or adapted for use in accessing other neural targets or other tissues throughout the body. Some examples include occipital nerves, peripheral nerve branches, nerves in the high cervical area, nerves in the thoracic area, and nerves in the lower sacral area.

A variety of pain-related conditions are treatable with the systems, methods and devices of the present invention. In particular, the following conditions may be treated:
1) Failed Back Surgery syndrome
2) Chronic Intractable Low Back Pain due to:
   A) Unknown Etiology
   B) Lumbar facet disease as evidenced by diagnostic block(s)
   C) Sacroiliac Joint disease as evidenced by diagnostic block(s)
   D) Spinal Stenosis
   E) Nerve root impingement—non-surgical candidates
   F) Discogenic Pain—discography based or not
3) Complex Regional Pain Syndrome
4) Post-Herpetic Neuralgia
5) Diabetic Neuropathic Pain
6) Intractable Painful Peripheral Vascular Disease
7) Raynaud's Phenomenon
8) Phantom Limb Pain
9) Generalized Deafferentation Pain Conditions
10) Chronic, Intractable Angina
11) Cervicogenic Headache
12) Various Visceral Pains (pancreatitis, etc.)
13) Post-Mastectomy Pain
14) Vulvodynia
15) Orchodynia
16) Painful Autoimmune Disorders
17) Post-Stroke Pain with limited painful distribution
18) Repeated, localized sickle cell crisis
19) Lumbar Radiculopathy
20) Thoracic Radiculopathy
21) Cervical Radiculopathy
22) Cervical axial neck pain, “whiplash”
23) Multiple Sclerosis with limited pain distribution
   Each of the above listed conditions is typically associated with one or more DRGs wherein stimulation of the associated DRGs provides treatment or management of the condition.

Likewise, the following non-painful indications or conditions are also treatable with the systems, methods and devices of the present invention:
1) Parkinson's Disease
2) Multiple Sclerosis
3) Demylenating Movement Disorders
4) Physical and Occupational Therapy Assisted Neurostimulation
5) Spinal Cord Injury—Neuroregeneration Assisted Therapy
6) Asthma
7) Chronic Heart Failure
8) Obesity
9) Stroke—such as Acute Ischemia
   Again, each of the above listed conditions is typically associated with one or more DRGs wherein stimulation of the associated DRGs provides treatment or therapy. In some instances, Neuroregeneration Assisted Therapy for spinal cord injury also involves stimulation of the spinal column.

It may be appreciated that the systems, devices and methods of the present invention may alternatively or additionally be used to stimulate ganglia or nerve tissue. In such instances, the condition to be treated is associated with the ganglia or nerve tissue so that such stimulation provides effective therapy. The following is a list of conditions or indications with its associated ganglia or nerve tissue:
1) Trigeminal Neuralgia (Trigeminal Ganglion)
2) Hypertension (Carotid Sinus Nerve/Glossopharangyl Nerve)
3) Facial Pain (Gasserian Ganglion)
4) Arm Pain (Stellate Ganglion)
5) Sympathetic Associated Functions (Sympathetic Chain Ganglion)
6) Headache (Pterygoplatine Ganglion/Sphenopalatine Ganglion)

It may also be appreciated that the systems and devices of the present invention may also be used to stimulate various other nerve tissue including nerve tissue of the peripheral nervous system, somatic nervous system, autonomic nervous system, sympathetic nervous system, and parasympathetic nervous system, to name a few. Various features of the present invention may be particularly suited for stimulation of portions of these nervous systems. It may further be appreciated that the systems and devices of the present invention may be used to stimulate other tissues, such as organs, skin, muscle, etc.

Although the foregoing invention has been described in some detail by way of illustration and example, for purposes of clarity of understanding, it will be obvious that various alternatives, modifications, and equivalents may be used and the above description should not be taken as limiting in scope of the invention which is defined by the appended claims.

What is claimed is:

1. A system for accessing a target location in an epidural space of a patient, the system comprising:
a sheath having a proximal end, a pre-curved distal end and a lumen having an inner diameter; and
a sheath support having a shaft configured to be disposed within the lumen of the sheath and a distal tip,
wherein the sheath support is sufficiently flexible to bend according to the pre-curved distal end of the sheath and
wherein the sheath support has a non-compliant outer diameter that maintains the inner diameter of the lumen of the sheath so as to resist kinking of the sheath.

2. The system of claim 1, wherein the distal tip is retractable through the lumen of the sheath and removable from the proximal end of the sheath.

3. The system of claim 1, wherein together the sheath and sheath support disposed therein is flexible.

4. The system of claim 3, wherein the sheath and sheath support disposed therein is advanceable through an epidural introducing needle.

5. The system of claim 1, wherein the sheath support shaft has an outer diameter that sufficiently matches the inner diameter of the sheath while allowing movement of the sheath support relative to the sheath.

6. The system of claim 1, wherein the sheath support shaft is comprised of a coil.

7. The system of claim 6, wherein a distal portion of the coil has a larger pitch than a proximal portion of the coil.

8. The system of claim 6, wherein the distal tip comprises a distal end cap molded to the coil.

9. The system of claim 8, wherein the distal end cap comprises an inner tubular shaft and an outer tubular shaft, wherein the inner and outer tubular shafts are concentrically positioned and joined at one end by the distal tip.

10. The system of claim 1, further comprising an elongate device adapted to be advanced through the sheath such that the curved distal portion of the sheath bends and guides the elongate device toward the target location as the elongate device is advanced therethrough.

11. The system of claim 10, wherein the elongate device comprises a lead, catheter, stylet, guidewire or tool.

12. The system of claim 1, wherein the target location comprises a spinal nerve.

13. The system of claim 1, wherein the target location comprises a dorsal root ganglion.

14. The system of claim 1, further comprising a retraction shield having a lumen, wherein the retraction shield is configured to be disposed within the lumen of the sheath while the sheath support shaft is disposed within the lumen of the retraction shield.

15. The system of claim 14, wherein the distal tip is at least partially retractable into the lumen of the retraction shield and together the sheath support and retraction shield are removable from the proximal end of the sheath.

16. A system as in claim 1, wherein the distal tip is configured to resist retraction into the lumen of the sheath until a threshold force is reached which causes the distal tip to at least partially retract into the lumen of the sheath.

17. The system of claim 16, wherein the distal tip at least partially collapses while it at least partially retracts into the lumen of the sheath.

18. The system of claim 16, wherein a portion of the sheath support shaft is configured to at least partially collapse while the distal tip at least partially retracts into the lumen of the sheath.

19. The system of claim 16, wherein the distal tip is comprised of a flexible polymer which changes shape while at least partially retracting into the lumen of the sheath.

20. The system of claim 16, wherein the distal tip has a ball shape.

21. The system of claim 16, wherein the distal tip has an atraumatic shape.

22. The system of claim 16, wherein the distal tip has a cutting tip, agent delivery tip, a vision tips, an electrical energy delivery tip, and/or a stimulation tip.

23. A method for accessing a target location in the epidural space of a patient, the method comprising:
advancing an introducer needle into the epidural space;
advancing a sheath and a sheath support disposed therein through the introducer needle and within the epidural space toward the target location, wherein the sheath support is sufficiently flexible to bend according to a pre-curved distal end of the sheath and wherein the sheath support has a non-compliant outer diameter that maintains the inner diameter of the lumen of the sheath so as to resist kinking of the sheath;
positioning the distal end of the sheath and sheath support disposed therein near the target location; and
retracting the sheath support into the sheath and removing the sheath support from a proximal end of the sheath leaving the distal end of the sheath near the target location.

24. The method of claim 23, wherein the target location comprises a dorsal root ganglion.

25. The method of claim 24, wherein the positioning step comprises positioning the distal end of the sheath and the sheath support disposed therein along a nerve root associated with the dorsal root ganglion.

26. The method of claim 24, wherein the positioning step comprises positioning the distal end of the sheath and the sheath support disposed therein within a foramen associated with the dorsal root ganglion.

27. The method of claim 23, further comprising inserting an elongate device through the sheath such that at least a portion of the device extends out of the distal end of the sheath toward the target location.

28. The method of claim 27, wherein the elongate device comprises a lead, catheter, guidewire, stylet, or tool.

29. The method of claim 27, wherein the elongate device comprises a lead having at least one electrode and the method further comprises delivering stimulation energy from at least one of the at least one electrode toward the target location.

30. The method of claim 29, wherein the target location comprises a dorsal root ganglion.

31. The method of claim 27, wherein the elongate device comprises an agent delivery device and the method further comprises delivering an agent to the target location.

32. The method of claim 31, wherein the target location comprises a dorsal root ganglion.

33. The method of claim 23, further comprising advancing the sheath support beyond the distal end of the sheath so that the distal tip atraumatically tunnels through a resistant area of the epidural space.

34. The method of claim 33, wherein the resistant area comprises a foramen and the tunneling creates additional space within the foramen.

35. The method of claim 33, further comprising advancing and retracting the sheath support to create additional tunneling force or friction along the resistant area.

36. The method of claim 23, wherein the sheath support has an atraumatic distal tip configured to resist retraction into the sheath while covering a distal end of the sheath, and wherein the retracting step further comprises applying a threshold force during retracting which overcomes the resistance allowing the atraumatic distal tip to at least partially retract into the lumen of the sheath.

37. The method of claim 23, further comprising advancing an elongate device through the sheath to perform a function at the target location, wherein the function includes neuromodulating, electrically stimulating, sensing, cutting, piercing, ablating, visualizing and/or delivering an agent.

38. The method of claim 37, wherein the elongate device comprises a lead having at least one electrode and the target location comprises a dorsal root ganglion, the method further comprising providing stimulation energy to the at least one of the at least one electrodes to selectively stimulate the dorsal root ganglion.
